(19) United States
(12) Patent Application Publication
(10) Pub. No.: US 2005/0250173 A1
(43) Pub. Date: Nov. 10, 2005

(54) Detection device, components of a detection device, and methods associated therewith

(76) Inventors: Charles Quentin Davis, Frederick, MD (US); Richard Cook, Derwood, MD (US); Rober Kirkor Matikyan, Rockville, MD (US); Manish Swarnaraj Kochar, Rockville, MD (US); Jason Charles Harley, Gaithersburg, MD (US); Jonathan Matthew Miller, Fairfax, VA (US); Jeffrey Arthur Daniels, Washington, DC (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/841,569

(22) Filed: May 10, 2004

(51) Int. Cl. C12Q 1/02; G01N 21/00

(52) U.S. Cl. 435/29; 435/4; 356/343

(57) ABSTRACT

A detection system may include a moveable tray configured to hold a multi-cell container of one or more reagents and/or one or more samples. A driving mechanism may be configured to reciprocate the tray in the first linear direction to agitate contents of the container, and may be configured to conduct electrochemiluminescence or other measurements on samples located in the container. The system may include an apparatus for retaining a container, a device for detecting the presence of a container, an apparatus for training a probe to locate and aspirate one or more reagents and/or one or more samples, a latching mechanism for moving parts in the system, and/or a positive displacement pump. A controller may be configured to control linear reciprocation of the tray to have one of a piecewise constant velocity profile and piecewise constant acceleration profile in which the number of piecewise constants does not exceed 24.

### TECHNICAL FIELD

The present invention relates to a detection device and, more particularly, to components of a detection device and methods associated with those components.

### BACKGROUND

Conventional detection systems may include fluidic systems for moving and mixing samples and reagents. In many applications, the samples and reagents may include complex matrices that may contain salts, air bubbles, and/or particulate matter that can reduce the performance or damage fluidic systems. It is desirable that fluidic systems used in biological detection systems are capable of handling such complex matrices. At the same time, it is desirable that fluidic systems have relatively low complexity so as to increase the reliability and robustness of the systems and reduce cost.

Some conventional biological detection systems employ multi-cell containers as sample and/or reagent carriers so as to allow for greater automation of assay procedures and to increase assay throughput. The multi-cell containers may include microplates, which are commercially available in various standardized sizes and formats (e.g., microplates can have several different flange systems forming the base of the plate), as specified by the Society for Biomolecular Screening (SBS). It is important that biological detection systems be able to correctly identify and/or interrogate specific wells on the sample carriers. Misalignment of sample carriers or instrument components can lead to interrogation of incorrect wells and spurious results and may also lead to instrument damage. Improved methods and devices for aligning sample carriers and instrument components are needed.

Some conventional detection devices use oscillatory motion in a linear fashion to agitate a multiplicity of samples contained in a multi-assay plate. However, these conventional devices do not attempt to control the amount of harmonic content resulting from the linear shaking. In particular, controlling the amount of energy in the first 7 harmonics can be used, for example, to achieve higher amplitudes, reduce vibration in the rest of the detection system, and/or prevent splashing of samples from the multi-assay plate. Furthermore, it may be desirable to minimize software and/or controller impact by using a series of motion profiles that control only velocity and acceleration to control the harmonic content of the shaking.

When biological detection systems use containers that are placed in the system by human operators, there exists the possibility that the operator will install the container improperly. In particular, the operator can install a container backwards. With microplates, the SBS standards do not specify a key that could be used to ascertain correct orientation. One method that is commonly used to prevent this human error is to label the microplate receptacle. While this labeling reduces errors, it cannot prevent them. Alternatively, a biological detection system can restrict usage to a subset of possibly custom microplates and mechanically key the system to prevent both the installation of a misoriented acceptable microplate and the installation of non-approved microplates. However, such a system would prevent use of otherwise-acceptable microplates. Alternatively, orientation could be ascertained through the use of a keying feature such as a barcode label that is placed on the microplate. This method has the disadvantages of possibly requiring additional hardware to detect the label and requiring a step of applying the label. Thus, it may be desirable for a biological detection system to ensure the correct orientation of containers through the use of custom sample containers, while still permitting usage of non-custom SBS-compliant microplates.

Some conventional biological detection systems do not secure internal components during transport or other non-operating conditions. It may be desirable for biological detection systems to include mechanisms and methods for securing internal components during transport or other non-operating conditions. For example, a portable unit that may be used for biological testing during a military operation, a terrorist attack, or other catastrophic event may be subject to conditions greatly different from those in a laboratory. Thus, it may be desirable for the detection device to be able to withstand these types of conditions.

### SUMMARY OF THE INVENTION

According to an exemplary aspect of the invention, an apparatus may retain a container in a biological detection device, with the container being configured to hold a plurality of at least one of samples and reagents. Throughout the disclosure, the term “container” generally refers to any multi-cell carrier that contains at least one sample and/or at least one reagent. The container may have any one of a plurality of different predetermined flange heights. The apparatus may include a first positioning block comprising a retractable first positioning arm and at least one retaining ledge on the first positioning arm, and a second positioning block having at least one additional retaining ledge. The first and second positioning blocks may be arranged to receive the container, and the first positioning arm may be adapted to selectively apply a biasing force to the container to position the container under the at least one additional retaining ledge.

In one embodiment, the second positioning block may further include a retractable slide. The slide may be configured to apply a second biasing force to the container in a direction substantially opposite to the biasing force. The second biasing force may be lesser in magnitude than the biasing force. When the biasing force is removed, the second biasing force may eject the container from the at least one additional retaining ledge. The second positioning block may further include a retractable second positioning arm. At least one of the second plurality of retaining ledges may be on the second positioning arm, and the second positioning arm may be configured to apply a third biasing force to the container that is lesser in magnitude than the first biasing force.

In another embodiment, the apparatus may further include a base member and a tray configured to translate in a first linear direction relative to the base member between a retracted position and an extended position. The tray may include the first and second positioning blocks. The first biasing force may be removed when the tray is in the extended position. The apparatus may further include a driving mechanism configured to translate the tray in the first linear direction. The driving mechanism may also be configured to reciprocate the tray in the first linear direction so as to agitate contents of the container. The driving mechanism may include, for example, a motor such as, for example, a stepping motor.

According to another aspect of the invention, a biological detection system may include a base member, a tray linearly movable with respect to the base member between an extended position and a retracted position, a first sensor on the base member, a second sensor on the base member, and an apparatus configured to conduct electrochemiluminescence measurements. The first sensor may be configured to detect whether the tray is in the retracted position, and the second sensor may be sensor configured to detect whether the tray is holding a container.

In one embodiment, the tray may include a first indicator and a second indicator. The first sensor may be configured to detect the first indicator when the tray is in the retracted position, and the second sensor may be configured to detect the second indicator when the tray is holding a container. The first and second sensors may include electro-optic sensors, and the first and second indicators may include vanes extending from the tray.

According to another embodiment, the tray may include a first indicator and a second indicator. The first sensor may be configured to detect the first indicator when the tray is in the retracted position, and the second sensor may be configured to detect the second indicator when the tray is not holding a container. The first and second sensors may include electro-optic sensors, and the first and second indicators may include vanes extending from the tray.

In accordance with another aspect of the invention, an apparatus for training a probe to locate and aspirate one or more reagents and/or one or more samples may include a first surface, a probe having a probe axis, and a motion control system for controlling relative movement of the probe with respect to the first surface in at least a first direction along the probe axis and at least a second direction not parallel to the probe axis. The probe may be movable relative to the first surface. The apparatus may further include a training object having an electrically-conductive training surface and a member contacting the first surface, means for applying an electrical signal between the probe and the training object via the first surface, and means for measuring a change in said electrical signal.

In an embodiment, the information content of the electrical signal results from a measurement of a DC potential, an AC potential, a DC current, an AC current, a DC charge, or an AC charge.

In another embodiment, the apparatus further comprises at least one alignment feature on the training object sized in accordance with a fabrication tolerance of the apparatus. The knowledge of at least one of a location and a size of the alignment feature in three or fewer dimensions may be refined from an original fabrication tolerance by using (i) the motion control system to move at least one of the probe and training object, and (ii) the electrical signals generated when the probe and aspects of the alignment feature contact one another.

Some embodiments may further include at least one alignment feature on the training object sized in accordance with a fabrication tolerance of the apparatus. The knowledge of at least one of a location and a size of the alignment feature in three or fewer dimensions may be refined from an original fabrication tolerance by using (i) the motion control system to move at least one of the probe and training object, and (ii) the electrical signals generated when the probe and aspects of the alignment feature are in close proximity to one another.

According to yet another aspect of the invention, a biological detection system may include a base member, a tray configured to translate in a first linear direction relative to the base member and to hold a container, a driving mechanism configured to reciprocate the tray in the first linear direction so as to agitate contents of the container, and an apparatus configured to conduct electrochemiluminescence measurements.

In one embodiment, the driving mechanism may include a motor having an output shaft, and the system may further include a belt associated with the output shaft and forming a linear drive path for the tray and . The output shaft may be arranged at a first end of the drive path, and a wheel may be associated with the belt at a second end of the drive path. The belt may have two substantially parallel belt portions extending from the output shaft to the wheel. The tray may be attached to one of said two belt portions, and a counterweight may be mounted to the other of said two belt portions such that the counterweight is configured to linearly translate in a direction opposite to a translation direction of the tray.

According to one embodiment, the weight of the counterweight may be greater than 70% of the weight of the tray and less than 120% of the weight of the sum of the tray and the maximum expected weight of the container with at least one of a reagent and sample. In some embodiments, the counterweight may be substantially the same weight as the tray.

According to an embodiment, the driving mechanism may reciprocate the tray in accordance with a trapezoidal motion profile, wherein each wavelength of the profile has an increasing positive velocity component, a constant positive velocity component, a decreasing positive velocity component, a decreasing negative velocity component, a constant negative velocity component, and an increasing negative velocity component. In one embodiment, the wavelength may also include at least one constant zero velocity component.

In some embodiments, the driving mechanism includes a motor having an output shaft, a bearing mounted on the output shaft, and a power transfer mechanism mounted on the output shaft. The bearing position may be closer to a body of the motor than the power transfer mechanism. The bearing may resist greater than 50% of the linear force applied to the motor shaft via the power-transfer mechanism.

In accordance with still another aspect, a biological detection system may include a latching mechanism for a movable member. The movable member may be configured to translate in a linear direction relative to a base member between a retracted position and an extended position. The latching mechanism may include a latching member configured to latch the movable member in the retracted position and a spring-biased member configured to urge the movable member in a direction away from the retracted position. The latching member may be movable between a latching position and an unlatching position.

In an embodiment, the biological detection system may further comprise at least one additional movable member and an additional latching mechanism associated with each additional movable member. The movable member and each of the at least one additional movable members may be movable in a direction not parallel to one another. Each additional latching mechanism may include a latching member configured to latch the respective additional movable member in the retracted position, wherein the latching member may be movable between a latching position and an unlatching position. Each additional latching mechanism may also include a spring-biased member configured to urge the respective additional movable member in a direction away from the retracted position.

In another embodiment, the biological detection system may further include at least one additional movable member, wherein the movable member and each of the at least one additional movable members may be movable in a direction not parallel to one another. The latching member may be configured to latch one of the at least one additional movable member.

In an embodiment, the latching mechanism may further comprise a solenoid configured to move the latching member between the latching position and the unlatching position.

In an exemplary aspect of the invention, a positive displacement pump may include a reagent supply line, a pump interface line from which the pump aspirates and dispenses fluid, a storage line fluidly connectable with a pump chamber, and means for selectively connecting the storage line to the reagent supply line or the pump interface line. In some embodiments, the means for selectively connecting the storage line may be a 3-way valve.

In another exemplary aspect of the invention, a method of retaining a container having any one of a plurality of different predetermined flange heights may include retracting a first positioning arm, placing the container in a tray, translating the tray along a translation path, engaging the container from a first direction with the first positioning arm, engaging the container from a second direction with a second positioning block, wherein the second direction may be opposite to the first direction, and applying a biasing force in the first direction to the container to position the container under at least one retaining ledge.

In an embodiment, the method may further include applying a second biasing force to the container in a direction opposite to the biasing force. The second biasing force may be lesser in magnitude than the biasing force. According to one embodiment, the method may further include removing the first biasing force, and ejecting the container from the at least one of the second plurality of retaining ledges via the second biasing force. The method may further comprise translating the tray in a first linear direction between a retracted position and an extended position. The first positioning arm may be retracted when the tray is in the extended position. In the method, the first biasing force may be removed when the tray is in the extended position.

In one embodiment, the method may further include reciprocating the tray in a first linear direction so as to agitate contents of the container.

In accordance with still another aspect, a method of determining a status of a movable tray may include detecting whether a tray is in a retracted position with a sensor on the base member, and detecting whether the tray is holding a container with a sensor on the base member.

In an embodiment, the detecting of whether a tray is in a retracted position may include detecting a first indicator extending from the tray when the tray is in the retracted position. The detecting of whether the tray is holding a container may include detecting a second indicator extending from the tray when the tray is holding a container.

In another embodiment, the detecting of whether a tray is in a retracted position may include detecting a first indicator extending from the tray when the tray is in the retracted position. The detecting of whether the tray is holding a container may include detecting a second indicator extending from the tray when the tray is not holding a container.

According to another aspect of the invention, a method of training a probe along a probe axis to locate and aspirate one or more reagents and/or one or more samples within a biological detection device, wherein the probe has a probe axis, may include moving one of the probe and a training object along at least one additional axis, different from the probe axis, to within an initial estimate of an alignment feature and moving the probe along the probe axis into the alignment feature until the probe is sufficiently close to the training object that an electrical signal is generated.

In one embodiment of the method, each of the at least one additional axis and said probe axis are not parallel to one another.

In yet another aspect of the invention, a method of training a probe along at least one axis not parallel to a probe axis to locate and aspirate at least one reagent and/or at least one sample within a biological detection device may include moving one of the probe and a training object along at least one additional axis differing from the probe axis, so that the probe and the training object are within an initial estimate of an alignment feature along said at least one additional axis. The method may further include moving the probe along the probe axis into the alignment feature until the probe is below an uppermost surface of the alignment feature, moving one of the probe and the training object along a training axis in a first direction and a second direction opposite to the first direction until the probe is sufficiently close to the training object in each of the first and second directions that electrical signals are generated, and determining a center point of the alignment feature along the training axis.

In some embodiments, the determined center point may be used in a method of training a probe along a probe axis to locate and aspirate one or more reagents and/or one or more samples within a biological detection device, wherein the probe has a probe axis. The method may include moving one of the probe and a training object along at least one additional axis, different from the probe axis, to the determined center point, and moving the probe along the probe axis into the alignment feature until the probe is sufficiently close to the training object that an electrical signal is generated.

In yet another aspect, a method of training a probe along at least two axes not parallel to a probe axis to locate at least one of a reagent and a sample within a biological detection device may include (i) moving one of the probe and a training object along all axes to be trained, different from the probe axis, so that the probe and the training object are within an initial estimate of an alignment feature along the axes to be trained, (ii) moving the probe along the probe axis into the alignment feature until the probe is below an uppermost surface of the alignment feature, (iii) moving either the probe or the training object along one of the axes to be trained alternately in both possible directions until the probe is sufficiently close to the training object in each of the directions that electrical signals are generated, and (iv) computing and then moving the probe to an estimate of the center point of the alignment feature. The method may further include (v) repeating steps (iii) and (iv) for all axes to be trained, and (vi) repeating step (v) until either (a) the change in the estimate of the center point of the alignment feature is sufficiently small, or (b) the desired number of iterations of (v) has occurred

According to yet another aspect of the invention, a method of training a probe along a probe axis to locate and aspirate one or more reagents and/or one or more samples within a biological detection device, wherein the probe has a probe axis, may include moving one of the probe and a training object along at least one additional axis, different from the probe axis, to within an initial estimate of an alignment feature and moving the probe along the probe axis into the alignment feature until the probe is sufficiently close to the training object that an electrical signal is generated. The initial estimate may be replaced with the center point as determined by steps (i) to (vi) above.

In still another aspect of the invention, a biological detection method may include reciprocating a tray relative to a base member in a first linear direction so as to agitate contents of a container and conducting electrochemiluminescence measurements on samples located in the container.

According to one exemplary embodiment of the biological detection method, the reciprocating comprises driving a belt with a drive mechanism. The method may further include counterbalancing a weight of the tray with a counterweight coupled to a belt and reciprocating the counterweight in a second linear direction opposite to the first linear direction of the tray.

In one embodiment of the biological detection method, the drive mechanism reciprocates the tray in accordance with a trapezoidal motion profile, wherein each wavelength of the profile has an increasing positive velocity component, a constant positive velocity component, a decreasing positive velocity component, a decreasing negative velocity component, a constant negative velocity component, and an increasing negative velocity component.

In some embodiments, the six velocity components may have approximately equal durations. According to an embodiment, the wavelength may include at least one constant zero velocity component.

In another exemplary aspect of the invention, a method of latching a movable member may include translating the movable member in a linear direction relative to a base member between a retracted position and an extended position, latching the movable member in the retracted position, and urging the latched movable member in a direction away from the retracted position.

In some embodiments, the method of latching may occur with (i) electrical power using the biological detection system's controller, or (ii) without electrical power using the biological detection system's operator, wherein the operator is not required to use tools.

In still another aspect, a method of unlatching a movable member in a biological detection system may include moving the movable member in a direction away from the extended position, moving the latching member from the latching position to the unlatching position, and urging the movable member toward the extended position. In some embodiments, the moving of the movable member may free the latching mechanism to move from the latching position to the unlatching position.

According to yet another exemplary aspect of the invention, a method of operating a positive displacement pump may include selectively directing a flow of fluid from a reagent supply line to a storage line fluidly connectable to a pump chamber, and selectively directing a flow of fluid from the storage line to a pump interface line from which the pump aspirates and dispenses fluid.

In accordance with an embodiment, the method of operating a positive displacement pump may further comprise preventing the reagent directed to the storage line that is to be dispensed from the pump interface line from entering the pump chamber. The method may further comprise selectively directing fluid from the pump chamber to a waste line.

In another exemplary aspect of the invention, an apparatus may include a base member, a tray configured to translate in a first linear direction relative to the base member, wherein the tray may be configured to hold a container, a driving mechanism configured to reciprocate the tray in the first linear direction so as to agitate contents of the container, and a controller configured to control linear reciprocation of the tray to have one of a piecewise constant velocity profile and piecewise constant acceleration profile in which the number of piecewise constants does not exceed 24.

In some embodiments, the system may include an apparatus configured to conduct electrochemiluminescence measurements. According to one exemplary embodiment, the number of piecewise constants does not exceed 12. For example, in alternative embodiments, the number of piecewise constants may equal 3 or 2.

In accordance with still another exemplary aspect, a method of agitating samples may include reciprocating a tray relative to a base member in a first linear direction so as to agitate contents of a container, and controlling linear reciprocation of the tray to have one of a piecewise constant velocity profile and piecewise constant acceleration profile in which the number of piecewise constants does not exceed 24. According to an exemplary embodiment, the number of piecewise constants does not exceed 12. For example, in alternative embodiments, the number of piecewise constants may equal 3 or 2. In some embodiments, the method may include conducting electrochemiluminescence measurements on at least one sample located in a container.

In yet another exemplary aspect, a fluid handling station for a biological detection device may include a port configured to receive a probe, a chamber extending from the port to a closed end. The chamber may have a first portion connected to a second portion via a tapered region, and the first portion may have a cross-sectional area greater than that of the second portion. The fluid handling station may further include at least one fluid line configured to direct liquid reagent to the chamber. Each of the at least one fluid lines may be coupled to the chamber at substantially the same distance from the closed end and below the tapered region.

In an embodiment, the fluid handling station may further include an additional fluid line coupled to the chamber at a greater distance from the closed end than each of the at least one fluid line and an air line coupled to the chamber at a greater distance from the closed end than each of the at least one fluid line and the additional fluid line.

In another exemplary aspect of the invention, a method of ascertaining correct orientation of a container in a biological detection device may include inserting a container into a biological detection device, moving a probe used to aspirate and dispense fluids in the detection device to a predetermined location corresponding with a key associated with the container, detecting whether the key is at the predetermined location, and determining, based on said detecting, whether the container is correctly oriented in the detection system.

According to yet another exemplary aspect, an apparatus for venting one of a reagent bottle and a waste bottle in a biological detection device may include a two-state sealing mechanism built into either the bottle or a bottle cap, and an indicating mechanism to unambiguously indicate the state of the sealing mechanism. The two states may be (a) to connect the interior space in the bottle to exterior and (b) to close said connection.

In some embodiments of the venting apparatus, the indicating mechanism is visual. In some embodiments, the indicating mechanism may be an electrical signal that is fed back to another aspect of the biological detection system.

In an embodiment, the method of ascertaining correct orientation of a container may further include determining, based on said detecting, a type of container inserted in the detection system.

### BRIEF DESCRIPTION OF THE DRAWINGS

FIG. 1 is a schematic representation of one exemplary embodiment of a detection system in accordance with an aspect of the invention.

FIG. 2A is an isometric view of an exemplary container holding apparatus in a retracted position in accordance with an aspect of the invention.

FIG. 2B is an isometric view of an exemplary container holding apparatus in an extended position in accordance with an aspect of the invention.

FIG. 2C is a side view of an exemplary container holding apparatus in a retracted position in accordance with an aspect of the invention.

FIG. 2D is a cross-sectional view through line I-I of FIG. 2B in accordance with an aspect of the invention.

FIG. 3A is a side view of an exemplary status detection device in accordance with an aspect of the invention.

FIG. 3B is a side view of an exemplary status detection device in accordance with an aspect of the invention.

FIG. 4A is an isometric view of an exemplary training plate in accordance with an aspect of the invention.

FIGS. 4B-4D are plots illustrating an exemplary training method according to an aspect of the invention.

FIG. 5A is an isometric view of an exemplary latching mechanism in accordance with an aspect of the invention.

FIG. 5B is a top view of an exemplary latching mechanism and an exemplary movable member in accordance with an aspect of the invention.

FIG. 5C is a top view of another exemplary latching mechanism and an exemplary movable member in accordance with an aspect of the invention.

FIG. 5D is a side view of yet another exemplary latching mechanism and an exemplary movable member in accordance with an aspect of the invention.

FIG. 6 is a partial diagrammatic and schematic representation of an exemplary positive displacement pump in accordance with an aspect of the invention.

FIG. 7 is a cross-sectional view of an exemplary fluid handling station in accordance with an aspect of the invention.

FIGS. 8A and 8B are graphs illustrating the exemplary velocity and acceleration profiles in accordance with the invention.

FIG. 9 is an isometric view of an exemplary customized container in accordance with an aspect of the invention.

FIG. 10 is a cross-sectional side view of an exemplary apparatus for venting a bottle in accordance with an aspect of the invention.

### DETAILED DESCRIPTION

The features of the disclosure will be understood more fully from the following detailed description.

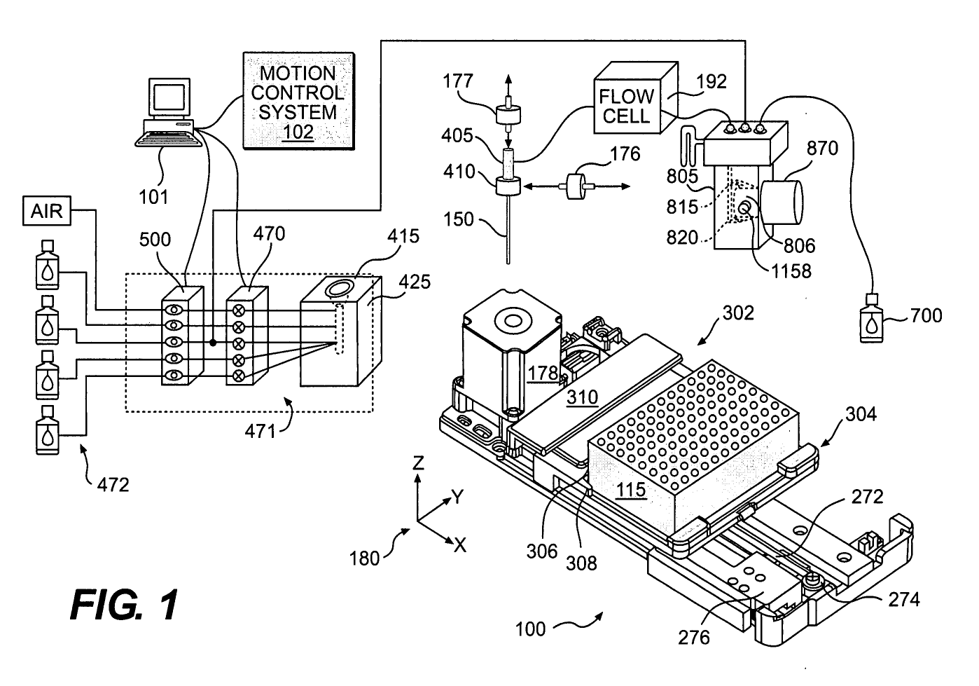

FIG. 1 is a schematic representation of an exemplary detection system, for example, a flow-cell based biological detection system. As depicted, overall operation of the detection system may be conducted under control of a computer system 101. Sample analysis occurs in flow cell 192, for example, a flow cell configured to measure radioactivity, optical absorbance, magnetic or magnetizable materials, light scattering, optical interference (i.e., interferometric measurements), refractive index changes, surface plasmon resonance, and/or luminescence (e.g., fluorescence, chemiluminescence and electrochemiluminescence). According to one aspect, the flow cell 192 is adapted for conducting electrochemiluminescence measurements. Exemplary electrochemiluminescence flow cells and methods for their use are disclosed in U.S. Pat. No. 6,200,531, the entire disclosure of which is hereby incorporated by reference. The operation of flow cell 192 may be controlled by the computer system 101, which may also receive assay data from the flow cell 192 and carry out data analysis.

Various automation systems may be employed such as a container alignment device and a pipettor (for example, a movable pipettor under automated control) for aspirating/dispensing fluids from one or more locations within the system. The container alignment device 100 depicted in FIG. 1 may be a simple one degree of freedom device that translates a container linearly from one position (typically where containers are added or removed from the biological detection system) to a second position (typically inside the detection system's housing), but may optionally be adapted to have additional degrees of freedom in the vertical direction or in the plane of the container. The system, however, is not limited to such a container alignment device and may utilize any system capable of transporting the container from a loading point to a point where the carrier is positioned for processing by the system. For example, a rotary system could be employed wherein the container is loaded on an arm that rotationally pivots about some point. The automated pipettor 405 shown in FIG. 1 may be capable of motion in two dimensions within a Cartesian coordinate system 180 through two independently controllable drive mechanisms 176, 177, for example, motors. Relative motion between the pipettor 405 and the container alignment device 100 in a third direction, not parallel to the other two dimensions, may be effected through a third independently controllable drive mechanism 178. The three directions of motion may be very close to mutually perpendicular, perhaps only having fabrication-related perturbations from perpendicularity, or may be distinctly non-perpendicular, perhaps due the lack of a requirement to move over all points in a rectangular box. Alternatively, motion control systems based on alternative coordinate systems may be used (e.g., one dimensional, two dimensional, polar coordinates, etc.). Operation of the automation systems may be controlled by a motion control subsystem. As depicted, the motion control subsystem 102 may receive instructions from the computerized system 101 which it then converts into appropriate control signals that direct one or more of the automation systems to perform the necessary steps to carry out the computerized system's instructions.

The exemplary flow-cell based biological detection system may also comprise a fluid handling station for introducing one or more reagents and/or one or more samples that may include gases and liquids. FIG. 1 depicts a fluid handling station 471 that may comprise flow control valves 470, reagent/gas detectors 500, and a fluid handling manifold 425. These devices may be independent fixtures fluidically connected (e.g., through flexible tubing) or may be integrated into a single system (as indicated by the dashed line). In an alternative embodiment, the location of valves 470 and sensors 500 along the fluidic lines may be switched so that sensors 500 are between reagent bottles 472 and valves 470.

The fluid handling manifold 425 may include an aspiration chamber employing a face-sealing configuration, for example, using an o-ring 415 arranged on a sealing surface of the manifold that may be adapted to achieve a fluidic seal between the manifold and a sealing surface 410 of the pipettor (e.g., a collar, flange, or the like). As depicted, the fluid handling manifold sealing surface is preferably located away from the reagent input lines (e.g., above the reagent lines' aspiration chamber entry points). Additionally, one or more of the reagent entry points can be positioned at predetermined heights within the aspiration chamber. For example, as depicted, the liquid reagent lines may be positioned beneath the gas reagent line to preclude contamination of the gas line. Reagent aspiration may be controlled by coordinating the selective actuation of one or more of the reagent valves 470 with the proper positioning of the pipettor and activation of the pump 870 so as to draw the reagents from the selected reagent bottles 472. Reagent detectors 500 may be employed to determine the presence and/or absence of reagent (e.g., whether one or more of reagent bottles 472 are empty), to determine the presence and/or absence of gaseous reagents (e.g., when air is used to segment fluids as they are aspirated), to determine/confirm the aspirated volume of a particular reagent, etc.

The detection system may be capable of precisely and accurately positioning the pipettor and the container so that the pipettor can be directed to aspirate/dispense fluids from a container and/or fluid handling station. Proper positioning may be accomplished through the use of alignment fixtures and/or through the proper training of the motion control system 102. To these ends, the system depicted in FIG. 1 utilizes positioning blocks 130, 140 arranged and configured to receive the container (here depicted as a microtiter plate) on a container alignment device 100 and to apply biasing forces to the container to precisely position the container 115 to a predetermined position within the system. The positioning blocks 302, 304 may be adapted and configured to precisely align the container 115 as it is being moved into the system by the container alignment device 100. Additionally, as indicated in FIG. 1, positioning blocks 302, 304 may also be configured to vertically retain/restrain the container in a predetermined position, for example, to prevent dislodgement of a container as a result of, for example, various forces experienced when the tray is reciprocated to agitate contents of the container 115 and/or vertical forces such as the fictional forces experienced when the pipettor is withdrawn from a pierced seal on the container.

The detection system may be capable of determining whether the container alignment device 100 is retracted or extended and/or whether the container 115 is present. Confirmation of the presence of container 115 and/or its proper positioning may be achieved by interrogating the detectors 200, 202 depicted schematically in FIG. 1. The motion control system may be trained or calibrated so as to compensate for manufacturing and/or assembly tolerances.

As shown in FIG. 1, the detection system may include a positive displacement pump 870 configured with a pump head manifold 805 that may be adapted to include a cleanout fluid path and plug 1158. Incorporation of the cleanout path and plug allows the pump's chamber (indicated by dashed lines) to be decontaminated in the event of failure of the pump's piston.

In an exemplary operation, container alignment device 100 loads container 115 (e.g., a microtiter plate) and properly aligns it within the detection system through the use of positioning blocks 302, 304. Detectors 200, 202 determine if the container is correctly positioned. Pipettor 405, under the control of motion control system 102, may be positioned in fluid handling manifold 425 and/or a well of container 115 so as to aspirate and/or dispense one or more samples and/or one or more reagents and introduce them into flow cell 192. The movement of fluids may be controlled through pump 870, and the selection of reagents aspirated from fluid handling manifold 425 may be controlled by valves 470 and sensors 500 operating so as to send an error message if a reagent line becomes empty. Optionally, pipettor 405 may also be used to combine one or more samples and/or one or more reagents into an incubation chamber (e.g., to carry out assay reactions prior to introduction of samples into flow cell 192). The incubation chamber may be, for example, a well of container 115 or an additional system component.

Assay measurements may be conducted on samples and/or assay reaction mixtures in flow cell 192. Computer system 101 may receive data and carry out data analysis. After completion of a measurement, the flow cell may be cleaned and prepared for the next measurement. The cleaning process may include the introduction of cleaning reagents into flow cell 192 by directing pipettor 405 and pump 870 to aspirate cleaning reagents from fluid handling manifold 425 or container 115.

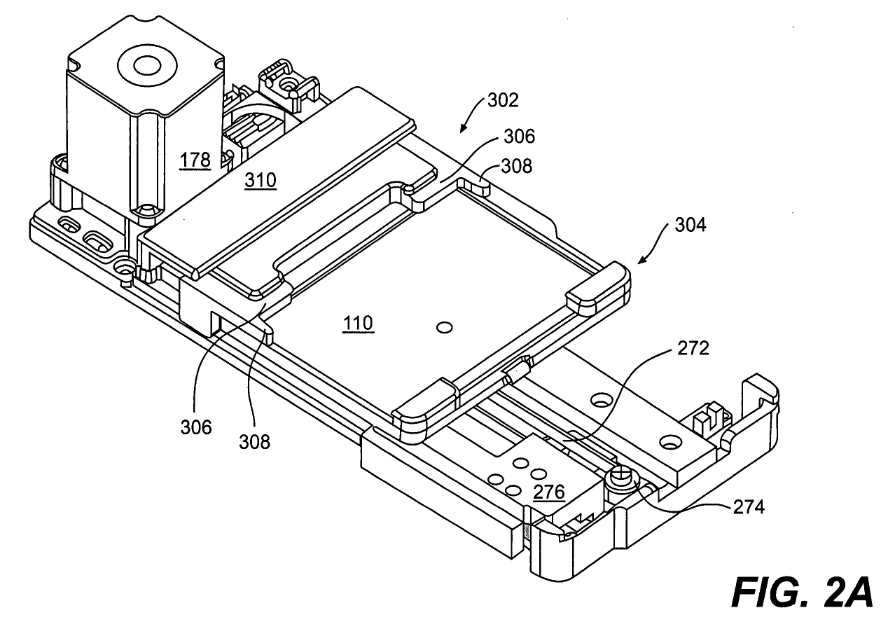

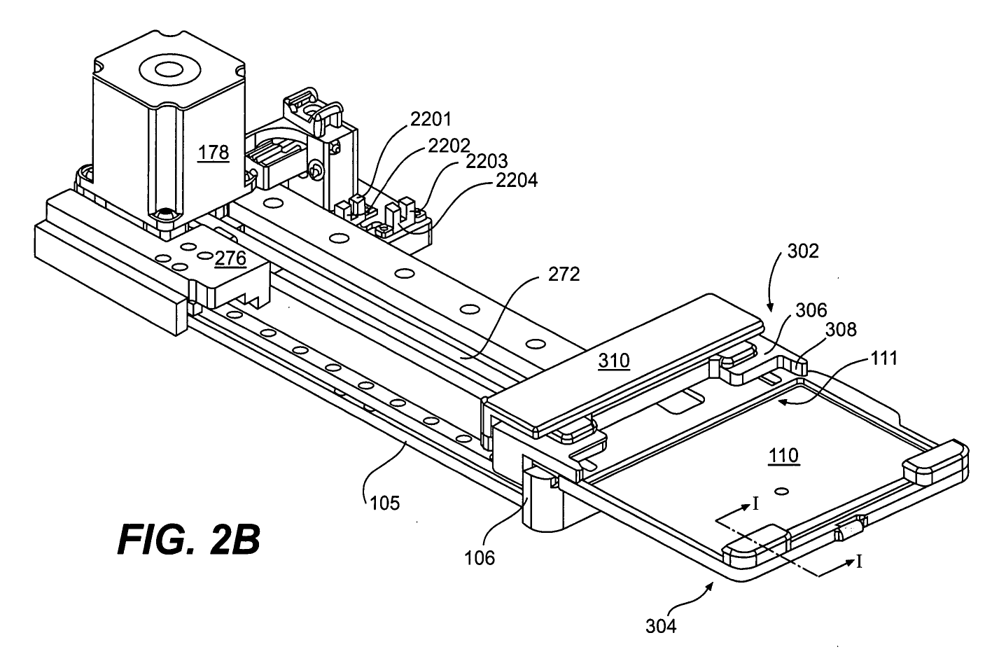

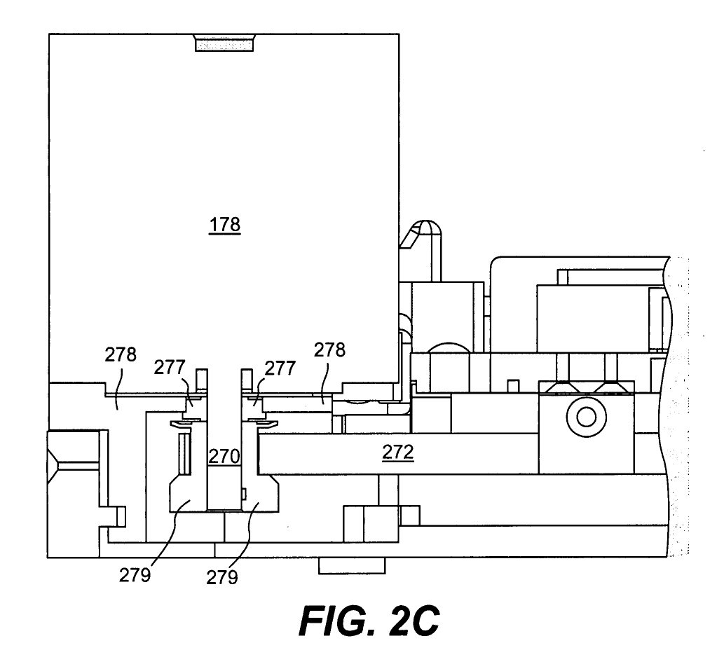

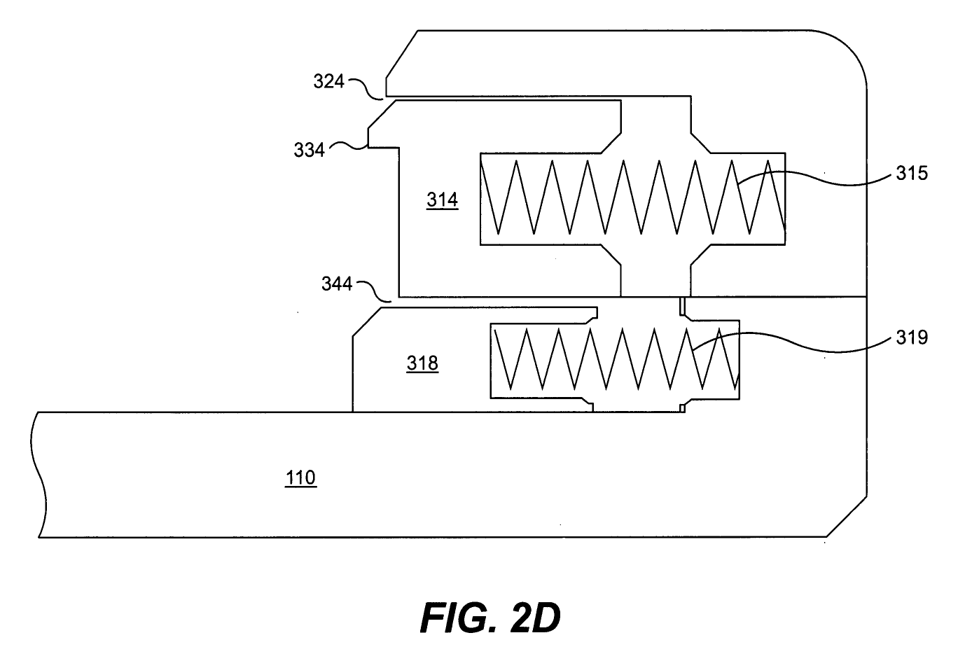

Referring now to FIGS. 2A and 2B, an exemplary container alignment device 100 is shown in retracted and extended positions, respectively. The container alignment device 100 may include a tray 110 linearly movable with respect to a base member 105. A drive mechanism 178, for example, a motor, such as a stepping motor, is provided for generating the relative movement between the tray 110 and the base member 105. The drive mechanism 178 includes an output shaft 270 (FIG. 2C), which may be mechanically coupled via a power-transfer mechanism 279 with a driven element, for example, a belt 272. The belt 272 may also be mechanically coupled with an idler wheel 274 to define a linear drive path, with the output shaft 270 and idler wheel 274 at opposed ends of the drive path.

Belt 272 is commonly under tension, for example, to reduce the likelihood of belt 272 from slipping on power-transfer mechanism 279. In some cases, the static force that belt 272 applies to the output shaft 270 via the power-transfer mechanism 279 may be large enough to damage the drive mechanism 178.

This damage may be prevented by using bearing 277. The static force that belt 272 applies to output shaft 270 via the power-transfer mechanism 279 is reacted by (1) bearing 277 and bearing mount 278 to the base member 105 and (2) the drive mechanism 178 to the base member 105. By loosening and re-tightening the drive mechanism 178 after belt 272 has been tensioned, the majority of the force can be reacted by bearing 277. By using bearing 277, the primary forces on the motor may be reduced to only the torque created by the separation between bearing 277 and power transfer mechanism 279. Said separation may be significantly smaller than the separation between bearing 277 and the top of drive mechanism 178. The force seen at the top of drive mechanism 178 may be approximately the ratio of two said separations (equaling a number less than 1) multiplied by the original force transferred to the output shaft 270 via the power-transfer mechanism.

The tray 110 may be mounted on the belt 272 so as to be linearly reciprocated by the drive mechanism 178 between the output shaft 270 and the idler wheel 274. A counterweight 276 may also be mounted on the belt 272 so as to be linearly reciprocated by the drive mechanism 178 between the output shaft 270 and the idler wheel 274. However, the tray 110 and, if present, counterweight 276 are arranged on the belt such that they are linearly reciprocated in opposite directions, as is apparent from FIGS. 2A and 2B. The counterweight 276 may have substantially the same weight as the tray 110, or perhaps the same weight as the tray 110 and the weight of container 115 loaded with one or more samples and/or one or more reagents. Counterweight 276 may reduce vibrations in the remainder of the biological detection system during reciprocation of the tray 110. Further, the amplitude of the reciprocation of tray 110 may be less dependent on the mass of the remainder of the biological detection system as well as the mounting (e.g., rubber feet) of the biological detection system to the surface on which it operates.

In one aspect, the drive mechanism 178 may be configured to reciprocate the tray 110 so as to agitate the contents of a container 115 being held by the tray 110. For example, the tray 110 may be reciprocated to agitate the contents of a cell in a container 115 so as to suspend a reagent in a fluid sample in preparation for conducting electrochemiluminescence measurements in the container or in an electrochemiluminescence flow cell.

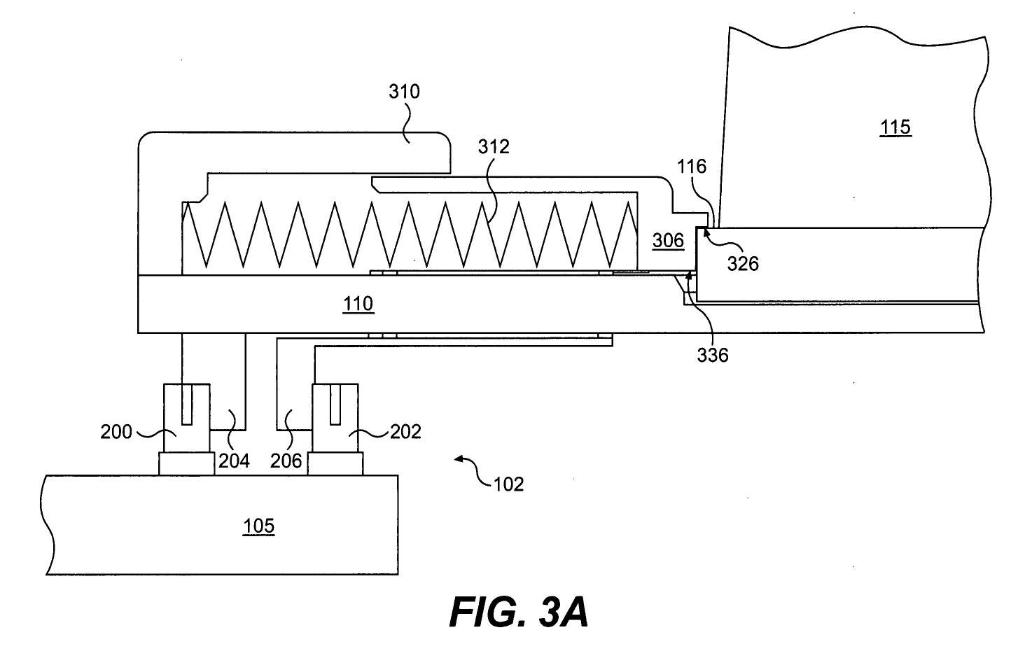

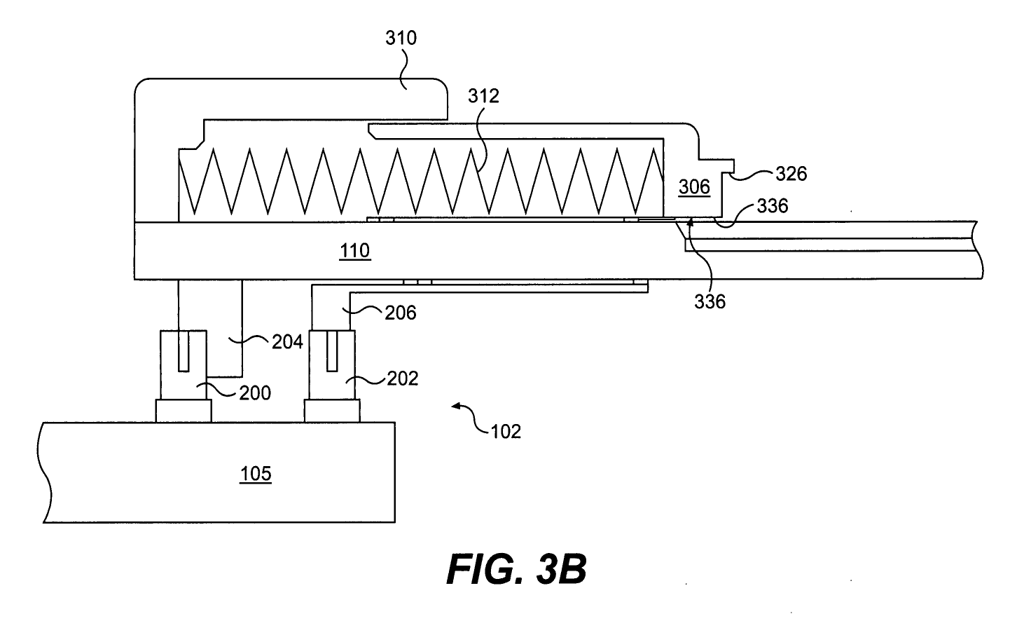

The alignment device 100 may be configured such that the container 115 may be secured for use by the detection system when the tray 110 is not near the extended position of FIG. 2B and such that a container 115 may be loaded onto the tray 110 when the tray 110 is in the extended position (e.g., a fully-extended position) of FIG. 2B. The alignment device 100 may include a first positioning block 302 and a second positioning block 304 opposite the first positioning block 302. The first positioning block 302 may include one or more positioning arms 306 retractable with respect to the tray 110. The positioning arms 306 may include tapered extension surfaces 308 configured to guide a container 115 onto the tray 110 and assist with alignment of the container 115 in a lateral direction with respect to the direction of linear reciprocation. The positioning arms 306 may be movable with respect to tray 110. The first positioning block 302 may be biased towards the second positioning block 304, for example, by a spring 312, between the first positioning block 302 and a spring retaining member 310 that is fixed to tray 110 as shown in FIG. 3.

Referring again to FIG. 2B, the first positioning block 302 may include a stop member 316 coupled to and extending from the positioning arms 306. When the tray 110 is driven to an extended position by the drive mechanism 178, the stop member 316 engages a stop member 106 of the base member 105. As the drive mechanism 178 continues to drive the tray 110, the bias of spring 312 is overcome, and the spring retaining member 310 and tray 110 move linearly relative to the positioning arms 306. Thus, the positioning arms 306 are effectively retracted relative to the tray 110, thus exposing a container alignment structure 111, for example, a shallow well in the tray 110 dimensioned to substantially match the predetermined size and shape of a container 115.

In an exemplary operation, a container 115 may be received by the alignment structure 111 of the tray 110. The drive mechanism 178 may then linearly drive the tray 110 from the fully-extended position toward a retracted position. As the tray 110 is driven toward the retracted position, the stop members 316, 106 begin separating, thus allowing the biasing force of the spring 312 to move the spring retaining member 310 away from the positioning arms 306. As a result, the positioning arms 306 are un-retracted relative to the tray 110 and may apply a clamping force to a container 115 on the tray 110 (FIG. 3). The positioning arms 306 may also include one or more retaining ledges 326, 336. The retaining ledges 326, 336 may be configured to receive a flange 116 of the container 115 and provide a retaining function with respect to the container 115 in a direction substantially perpendicular to the tray 110. The retaining ledges 326, 336 may be structured and arranged to correspond, for example, with flange sizes corresponding to standard containers 115 used in detection devices.

Referring now to FIG. 2D, the second positioning block 304 may include one or more positioning arms 314 urged by, for example, a spring 315 to apply a biasing force in a direction toward the first positioning block 302. The second positioning block 304 may also include a slide member 318 urged by, for example, a spring 319 to apply a biasing force in a direction toward the first positioning block 302. The positioning arms 314 and the slide member 318 may include stop members, which are well known in the art, in order to limit their range of motion. The second positioning block 304 may further include one or more retaining ledges 324, 334, 344, one or more of which may be on the positioning arms 314. The retaining ledges 324, 334, 344 may be configured to receive a flange 116 of the container 115 and provide a retaining function with respect to the container 115 in a direction substantially perpendicular to the tray 110. The retaining ledges 324, 334, 344 may be structured and arranged to correspond, for example, with flange sizes corresponding to standard containers 115 used in detection devices.

According to one aspect, the biasing forces of the springs 315, 319 associated with the second positioning block 304 may be substantially less than the biasing force of the spring 312 associated with the first positioning block 302. Referring again to FIG. 2B, when the tray 110 is driven to a fully-extended position and the positioning arms 306 are retracted, the biasing force of spring 312 on the container is removed. As a result, the biasing force of the spring 319 may urge the slide member 318 toward the first positioning block 302 and the container 115 from the second positioning block 304. For example, the container flange 116 may be urged from a position beneath one of the ledges 324, 334, 344, thus allowing simple and unobstructed removal of the container 115 from the tray 110.

Referring now to FIG. 3A, an exemplary alignment detection system 102 may include a first sensor 200 and a second sensor 202 on the base member 105. The detection system 102 may also include a first indicator 204 mechanically coupled to the tray 110 and a second indicator 206 mechanically coupled to at least one of the positioning arms 306.

The first sensor 200 and first indicator 204 may cooperatively operate to determine whether the tray 110 is in a retracted position. For example, the first sensor 200 may include a signal emitter member 2201 and a signal receiver member 2202 (FIG. 2B). In one aspect, the first sensor may be an opto-electronic sensor.

The first indicator 204 may include a vane extending from the tray 110. The first indicator 204 may be configured such that when the tray 110 is retracted, the first indicator is between the emitter member 2201 and the receiver member 2202. As a result, the first sensor 200 can detect the presence of the first indicator 204 and the controller 101 can determine that the tray is retracted to a desired position.

Similarly, the second sensor 202 and second indicator 206 may cooperatively operate to determine whether the tray 110 is holding a container 115. For example, the second sensor 204 may include a signal emitter member 2203 and a signal receiver member 2204 (FIG. 2B). In one aspect, the second sensor 202 may be an opto-electronic sensor. The second indicator 204 may include a vane extending from one of the positioning arms 306. The second indicator 206 may be configured such that when the tray 110 is correctly holding a carrier sample 115, the positioning arms 306 are retracted toward the spring retaining member 310, and the second indicator 206 is not between the emitter member 2203 and the receiver member 2204. As a result, the second sensor 202 does not detect the presence of the second indicator 206, and the controller 101 can determine that the tray 110 is correctly holding a container 115. Alternatively, as shown in FIG. 3B, the second indicator may be configured such that when the tray 110 is correctly holding a carrier sample 115, the positioning arms 306 are retracted toward the spring retaining member 310, and the second indicator 206 is between the emitter member 2203 and the receiver member 2204. As a result, the second sensor 202 detects the presence of the second indicator 206, and the controller 101 can determine that the tray 110 is correctly holding a container 115.

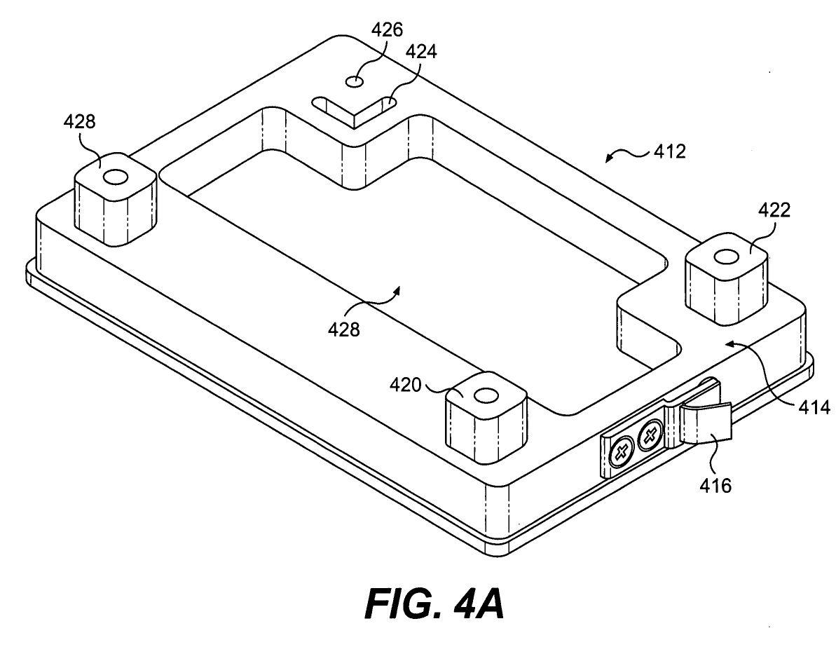

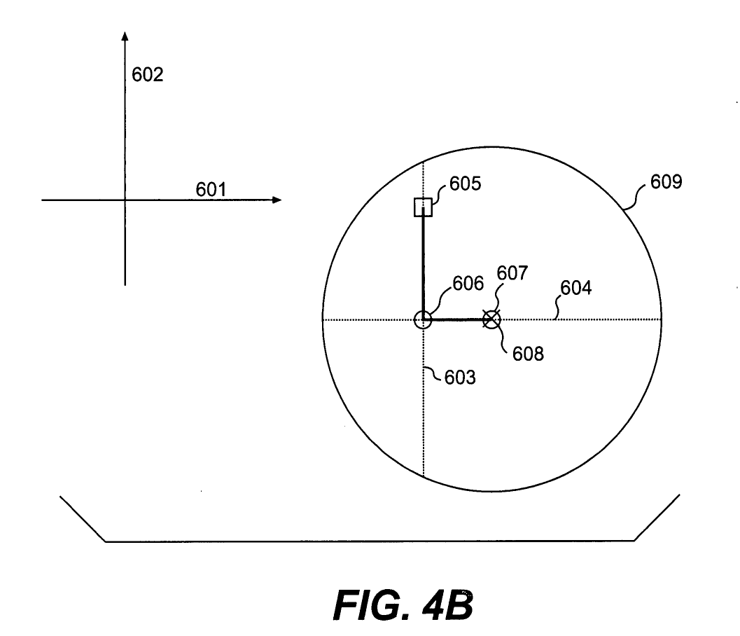

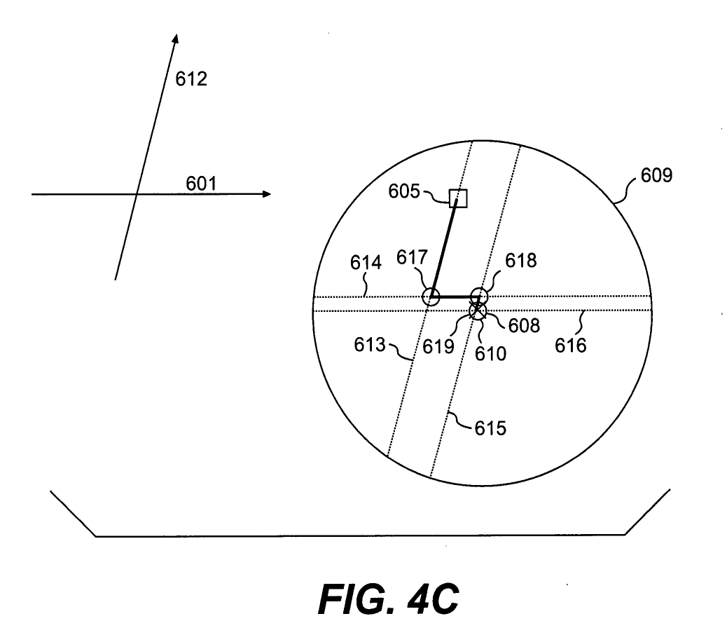

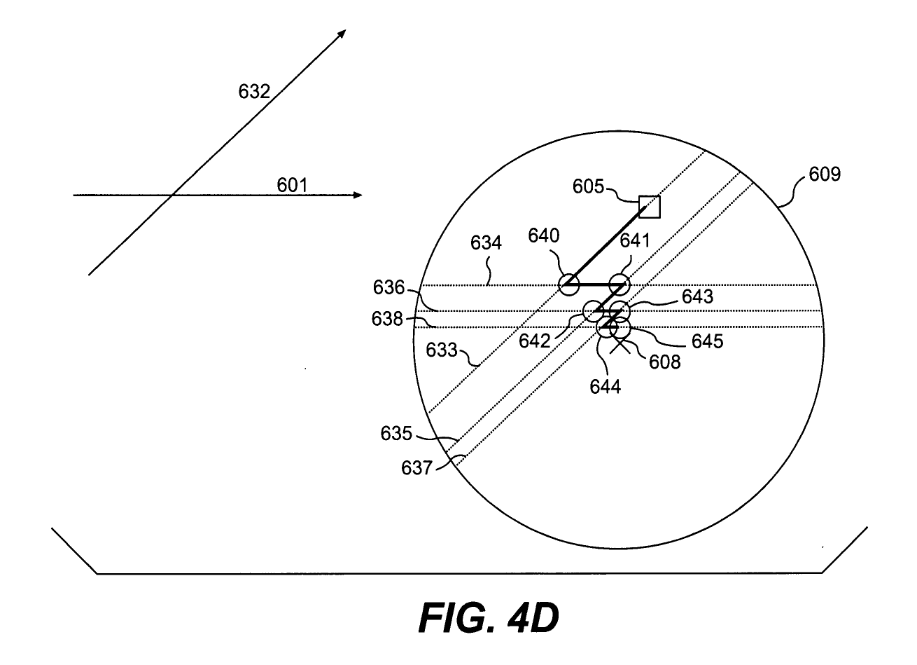

According to another exemplary aspect, a detection system may include a device and method of training, or calibrating, the position of the probe 150 relative to the tray 110. Referring now to FIG. 4a, a training apparatus may include a training object 412 having an electrically-conductive training surface 414. A grounding member 416 may be configured to contact a grounding surface associated with the base member 105. The training object 412 may include one or more alignment features 420, 422, 424, 426, 428. The configurations of the alignment features may be input or preprogrammed into the controller in order to provide a baseline for a training, or calibration, procedure.

An exemplary method of training the probe 150 may include moving the probe 150 relative to the training object 412 along an axis, for example, the x-axis, to within an initial estimate of one of the alignment features, for example, feature 420. The initial estimate may be determined by the controller 101 based on the input or preprogrammed data. The x-axis may be not parallel to the probe axis, which may correspond generally with the z-axis.

The method may further include moving the probe 150 along the probe axis (e.g., the z-axis) into the alignment feature 420 until the probe contacts the training object 412. A value associated with the contact point along the probe axis may be determined by the controller 101. The probe 150 may then be moved out of contact with the training object 412.

The method may then include moving the probe 150 along another axis, for example, the x-axis, in a first direction and a second direction opposite to the first direction until the probe 150 contacts the alignment feature 420 in each of the first and second directions. The controller 101 may determine values associated with the contact points in both the first and second directions along the x-axis. From these two values, the controller 101 can determine a center point of the alignment feature 420 along the x-axis.

The probe 150 may then be moved along another axis, for example, the y-axis, in a third direction and a fourth direction opposite to the third direction until the probe 150 contacts the alignment feature 420 in each of the third and fourth directions. The controller 101 may determine values associated with the contact points in both the third and fourth directions along the y-axis. From these two values, the controller 101 can determine a center point of the alignment feature 420 along the y-axis.

FIG. 4b shows an example of the training method described above. Axis 602 may correspond to the x axis; axis 601 to the y axis. Alignment feature 609 may be any of the features 420, 422, 426 or 428 on training object 412. Symbol 605 represents the initial estimate of the center of the alignment feature 608. In this example, axes 601 and 602 are perpendicular. Line 603 represents the path probe 150 took in the first and second directions, while symbol 606 represents the computed center. Line 604 then shows the path probe 150 took in the third and fourth directions, while 607 represents the computed center. Computed center 607 is extremely close to alignment feature's center 608, demonstrating good training.

FIG. 4c shows an example of the training method, where axes 601 and 612 are slightly not perpendicular. Lack of perpendicularity may occur by design or by assembly errors. In this case, repeating the steps above from the initial estimate 605 leads us through point 617 to 618, with probe paths 613 and 614. The process can be repeated with point 618 being the initial estimate, yielding point 620, which is close to alignment feature's center 608, demonstrating good training.

FIG. 4d shows an example of the training method, where axes 601 and 632 are not perpendicular. In this case, repeating the steps above from the initial estimate 605 leads us through point 640 to 641, with probe paths 633 and 634. A first repeat using 641 as the initial estimate leads us through point 642 to 643, with probe paths 635 and 636. A second repeat brings the computed center to 645 with probe paths 637 and 638, which is fairly close to the alignment feature's center 608. The number of repetitions in the process can be chosen in a number of ways, for example, by fixing the repetitions to a constant value, or by continuing to repeat until the difference between estimated center points on subsequent repetitions is below a threshold. One skilled in the art may also see that as the angle between the axes becomes smaller, the rate of convergence decreases. By knowing the designed angle and the fabrication tolerances, the rate of convergence could be estimated. Furthermore, improved methods can be made, for example, by noting the estimates 640, 642, and 644 fall on a straight line, as do estimates 641, 643, and 645. The intersection of these two lines is the alignment center 608.

The controller 101 may train, or calibrate, the probe 150 by comparing the determined center point of the alignment feature 420 with the input or preprogrammed data. It should be appreciated by one of ordinary skill in the art that the probe may be additionally or alternatively calibrated with respect to one or more of the other alignment features 422, 424, 426, 428 in a manner similar to that described above with respect to alignment feature 420. The knowledge that container 115 is a solid body enables a 6 degree of freedom calculation to compute the location of any feature on container 115 from the training information on, for example, alignment features 420, 422, and 428.

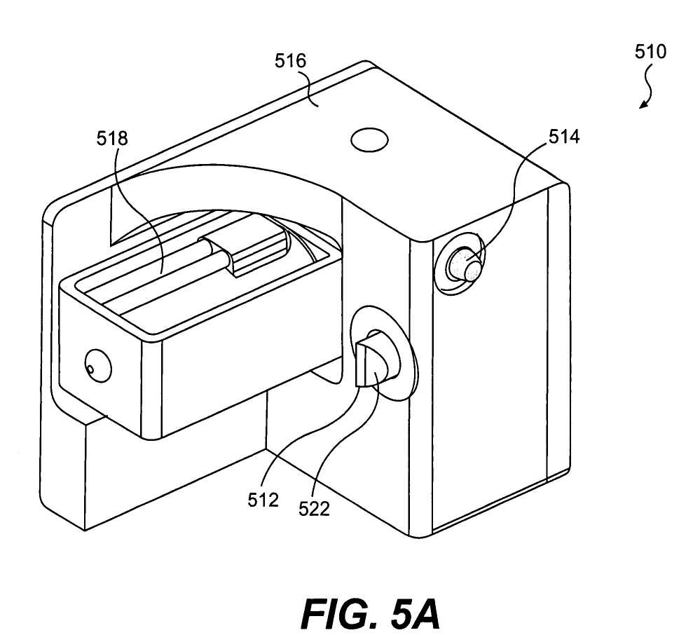

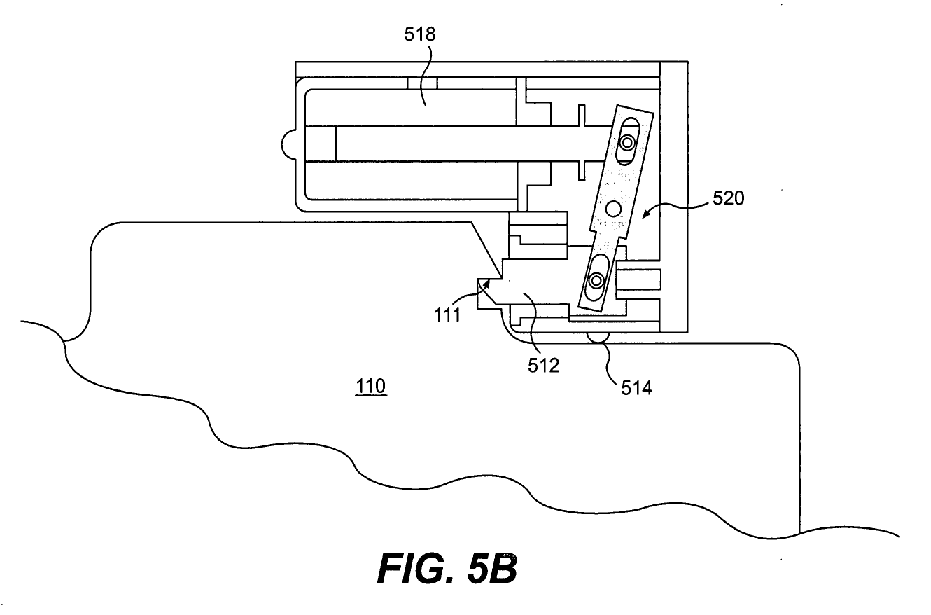

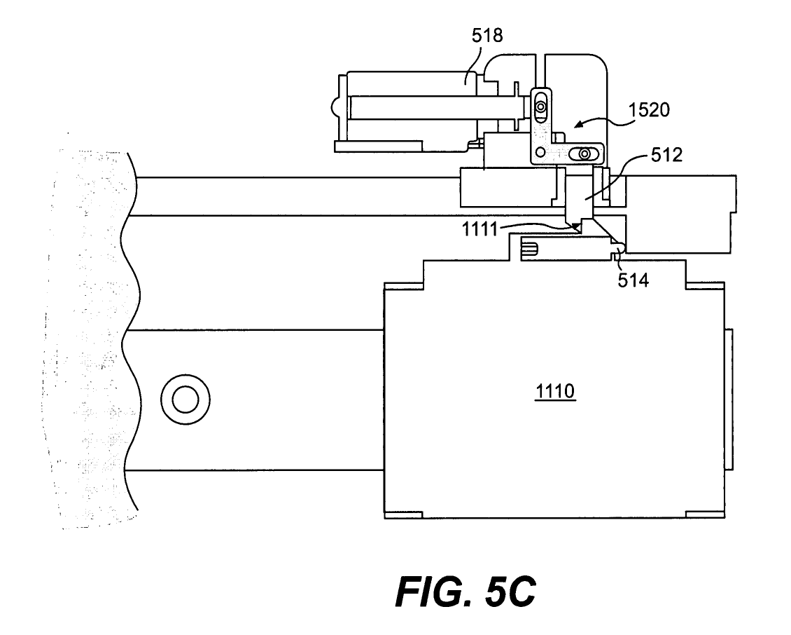

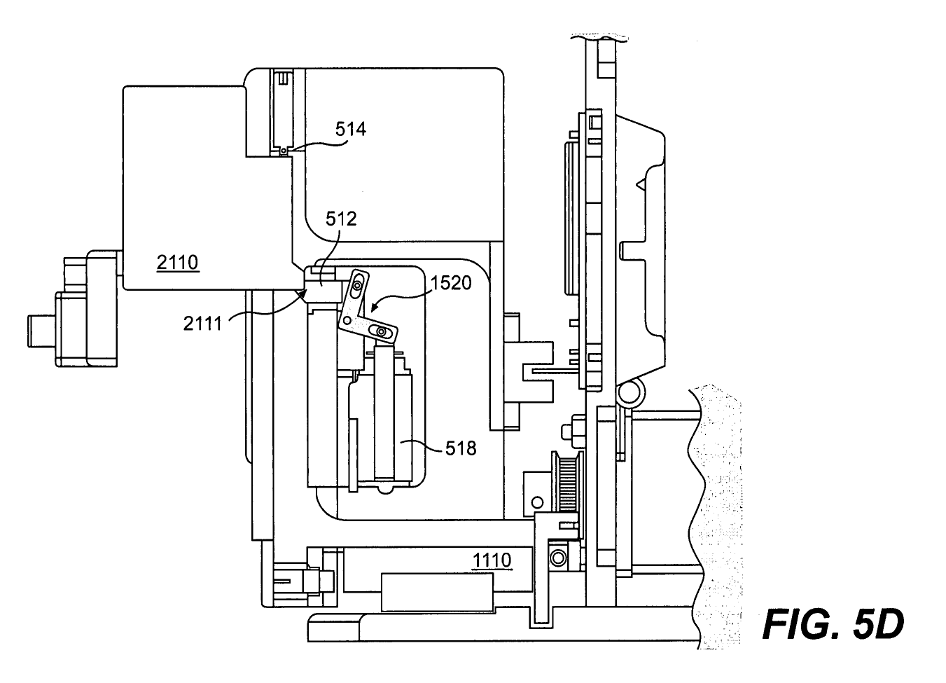

Referring now to FIGS. 5A-5D, an exemplary latching mechanism 510 for a movable member 110, 1110, 2110 may include a latching member 512 and a biasing member 514. The latching member 512 may extend from a housing 516 in a direction substantially perpendicular to the direction of linear reciprocation of the movable member 110, 1110, 2110. The latching member 512 may include a tapered surface 522 facing in a direction toward the movable member 110, 1110, 2110 when the movable member 110, 1110, 2110 is in an extended position. The biasing member 514, for example, a spring-biased member, may extend from the housing 516 in a direction perpendicular to the latching member 512, for example, in the direction of linear reciprocation of the movable member 110, 1110, 2110. The latching mechanism 510 may include an actuator 518, for example, a solenoid, configured to selectively move the latching member 512 between a latching position and an unlatching position. In one aspect, the latching member 512 is in the latching position, as shown in FIGS. 5A-5D, when the actuator 518 is not actuated and is moved to the unlatching position when the actuator 518 is actuated. Accordingly, movable member 110, 1110, 2110 may be latched in the absence of electrical power by, for example, the operator pushing movable member 110, 1110, 2110 into the retracted position, where the latching will happen automatically. Alternatively, the latching member 512 may be in the unlatching position when the actuator 518 is actuated and may be moved to the unlatching position when the actuator 518 is unactuated.

As depicted in FIG. 5B, the actuator 518 may be mechanically coupled to the latching member 512 via a 180° mechanical linkage 520. In FIG. 5B, the movable member is tray 110 that is linearly movable along the x-axis of the Cartesian coordinate system 180. As shown in FIGS. 5C and 5D, the actuator 518 may be mechanically coupled to the latching member via a 90° mechanical linkage 1520. In FIG. 5C, the movable member is a carriage 1110 that is linearly movable along the y-axis of the Cartesian coordinate system 180. In FIG. 5D, the movable member is a probe motion device 2110 that includes the probe 150 and is linearly movable along the z-axis of the Cartesian coordinate system 180. In one exemplary aspect, the probe motion device 2110 may be coupled to and movable with the carriage 1110. It should be appreciated that the actuator 518 may be structured and arranged such that it could be coupled to the latching member 512 with 0° or any other mechanical linkage arrangement.

In operation, as the tray 110 is retracted by the drive mechanism 178, the movable member 110, 1110, 2110 may engage the tapered surface 522 of the latching member 512 and may urge the latching member 512 toward an unlatched position. The drive mechanism 178 may continue to drive the movable member 110, 1110, 2110 toward a retracted position, and the movable member 110, 1110, 2110 may disengage the latching member 512, thus allowing the latching member to return to the latching position shown in FIGS. 5A-5D. The latching member 512 may then be positioned to engage a detent 111, 1111, 2111 on the movable member 110, 1110, 2110, as is well known by persons of skill in the art. In the retracted position of the movable member 110, 1110, 2110, the biasing member 514 may urge the movable member 110, 1110, 2110 toward an extended position such that the movable member 110, 1110, 2110 engages the latching member 512 and remains latched in the retracted position. The force biasing member 514 applies to movable member 110, 1110, 2110 may be arranged, for example, to prevent motion of movable member 110, 1110, 2110 due to the expected level of vibrations the detection system experiences during transit.

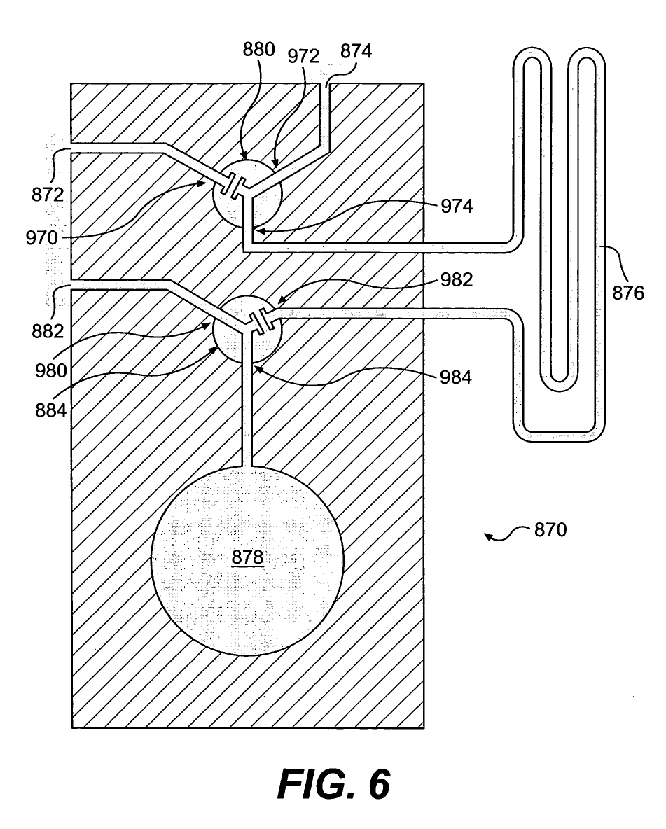

Referring now to FIG. 6, an exemplary positive displacement pump 870 for use with a detection system may include a reagent supply line 872 fluidly coupled with a reagent bottle 472 (FIG. 1) containing a reagent and a pump interface line 874 from which the pump 870 aspirates and dispenses fluid. The pump 870 may also include a storage line 876 fluidly connectable with a pump chamber 878. The pump 870 may also include a valve 880, for example, 3-way valve, having a first port 970, a second port 972, and a common port 974. The first port 970 may be fluidly connected to the reagent supply line 872, the second port 972 being fluidly connected to the pump interface line 874, and the common port 974 being fluidly connected to the storage line 876. The valve 880 may be operable to place either the reagent supply line 872 or the pump interface line 874 in fluid communication with the storage line 876. The storage line 876 may be dimensioned such that reagent that is to be dispensed from the pump interface line 874 may be drawn into the tube but does not enter the pump chamber 878.

The pump 870 may also include a waste line 882 and a second valve 884, for example, a 3-way valve, having a first port 980, a second port 982, and a common port 984, the first port 980 being fluidly connected to the waste line 882, the second port 982 being fluidly connected to the storage line 876, and the common port 984 being fluidly connected to the pump chamber 878. The valve 884 may be operable to place either the waste line 882 or the storage line 876 in fluid communication with the pump chamber 878.

In operation, controller 101 may operate the pump 870 to move the valve 884 to provide fluid communication between the pump chamber 878 and the waste line 882, so as to rid the pump chamber 878 of waste fluid. The controller 101 may also operate the pump 870 to move the valve 884 to provide fluid communication between the pump chamber 878 and the storage line 876. The pump 870 can then aspirate fluid into the storage line 876 via either the reagent supply line 872 or the pump interface line 874. Alternatively, the pump 870 may dispense fluid from the pump chamber 878 via the pump interface line 874.

In one aspect, the controller 101 may operate the pump 870 to aspirate reagent fluid into the pump chamber 878 from the fluid handling station 471 (FIG. 1) via the pump interface line 874 and the probe 150. The probe 150 may be moved to a cell of the container 115, and may begin dispensing fluid from the pump 870 via the pump interface line 874. As the pump chamber 878 empties, the controller 101 may operate the valve 970 to provide fluid communication between the reagent supply line 872 and the storage line 876 to refill with the storage line 876 with additional reagent fluid. This may eliminate the need to move the probe 150 from container 115 back to the fluid handling station 471 every time the pump chamber 878 is empty, which may happen for example when dispensing reagent into many cell in container 115. The controller 101 may then operate the valve 970 to provide fluid communication between the storage line 876 and the pump interface line 874 so as to resume dispensing of reagent fluid from the probe 150 via the pump interface line 874.

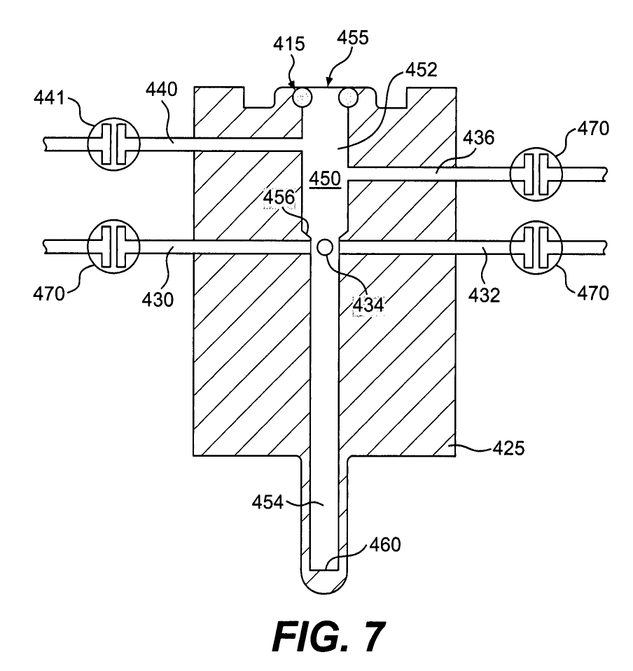

Referring now to FIG. 7, an exemplary fluid handling manifold 425 of a fluid handling station 471 (FIG. 1) may be used in a detection system, for example, a biological detection system. The fluid handling station 471 may be employed and configured, in accordance with an exemplary embodiment, to supply to a probe 150 (FIG. 1) the appropriate liquids through an access, or dispense, port 455 for aspiration into the flow cell. The fluidic probe 150, for example, a pipettor, pipe tip, syringe needle, cannula, or the like, may be used to access an aspiration chamber 450 of the fluid handling manifold 425 to aspirate the appropriate liquids.

As shown in FIG. 7, the aspiration chamber 450 may extend from the port 455 to a closed bottom end 460, and may include a first portion 452 and a second portion 454 connected to one another by a tapered region 456. The first portion 452 may have a larger cross-sectional area than the second portion 454. The aspiration chamber 450 may be connected to reagents, for example, through first, second, third, and fourth fluid lines 430, 432, 434, 436, respectively, and reagent valves 470 and to gas through gas line 440 and valve 441. As depicted in FIG. 7, the first, second, and third fluid lines 430, 432, 434 may enter the chamber 450 just below the tapered region 456 at substantially the same distance from the bottom end 460. The fourth fluid line 436 may enter the chamber 450 above the tapered region, as may the gas line 440.

A sealing surface 410 of the probe 150 may be sealed against a sealing surface 415 of the fluid handling manifold 425 to form a closed system upon insertion of the probe 150 into the chamber 450, for example, by utilizing a face sealing configuration located above the reagent inputs. The face sealing configuration may comprise, for example, a gasket or o-ring for forming a fluid and air tight seal. In one embodiment, the o-ring or gasket may be partially inset into a sealing surface of the manifold 425 leaving at least some portion of the o-ring, adequate for a compression seal, exposed above the surface of the manifold 425.

In operation, the probe 150 may be lowered to form the face seal in order to aspirate reagents. For example, the lowering may comprise compressing the sealing surface 410 against the sealing surface 415 so as to form a compression seal. According to one exemplary aspect, fluid, for example, liquid reagent, may be introduced into the chamber 450 via at least one of the fluid lines 430, 432, 434. Before insertion of the probe 105, the liquid reagent may extend from the bottom end 460 to a level below the tapered region 456 and below the first, second, and third fluid lines 430, 432, 434. When the probe 150 is inserted, the level of liquid reagent may rise to a level at the tapered region 456.

According to one exemplary aspect, the probe 150 may have a substantially circular cross-section with a maximum diameter of about 0.080 inches, and the second section 454 may have a substantially circular cross-section with a maximum diameter of about 0.095 inches. As shown in FIG. 7, the first portion 452 of the chamber 450 may have a diameter significantly larger than the second portion 454. When the probe 150 is removed from the chamber 450 while reagent is present, the first portion 452 and the tapered region 456 may prevent liquid from “walking up” the wall of the first portion 452 of the chamber 450, for example, by surface tension and/or capillary action of the liquid. This may prevent liquid reagent, which may have a significant salt content, from reaching the sealing surfaces 410, 415 of the probe 150 and the manifold 425. This, in turn, may assist with maintaining a compression seal between the probe 150 and the manifold 425.

During aspiration of reagents, the tip of probe 150 may be lower than the fluid lines 430, 432, 434, and 436, so that the flow of reagents may efficiently clean the probe surface and wash away any previous reagent or sample that were held in the aspiration chamber 450 or was located on the outside of the probe for example from sample or reagent located in container 115. For example, fluid line 436 may supply a reagent that is comprised substantially of pure water, optionally containing soap and/or an anti-microbial agent. The gas line 440 is preferably arranged sufficiently above the fluid lines 430, 432, 434, 436 in order to maintain a vertical separation between the gas line 440 and the fluid lines 430, 432, 434, 436. This may reduce or eliminate the contamination of the gas line 440 with liquid reagents. It also allows the aspiration of a bolus of air into the probe to be used to clear excess reagent from aspiration chamber 450 and/or to prevent mixing of reagents in the probe or subsequent fluid lines (i.e., by separating the reagents in the fluid lines into so-called “slugs” of fluid separated by boluses of air).

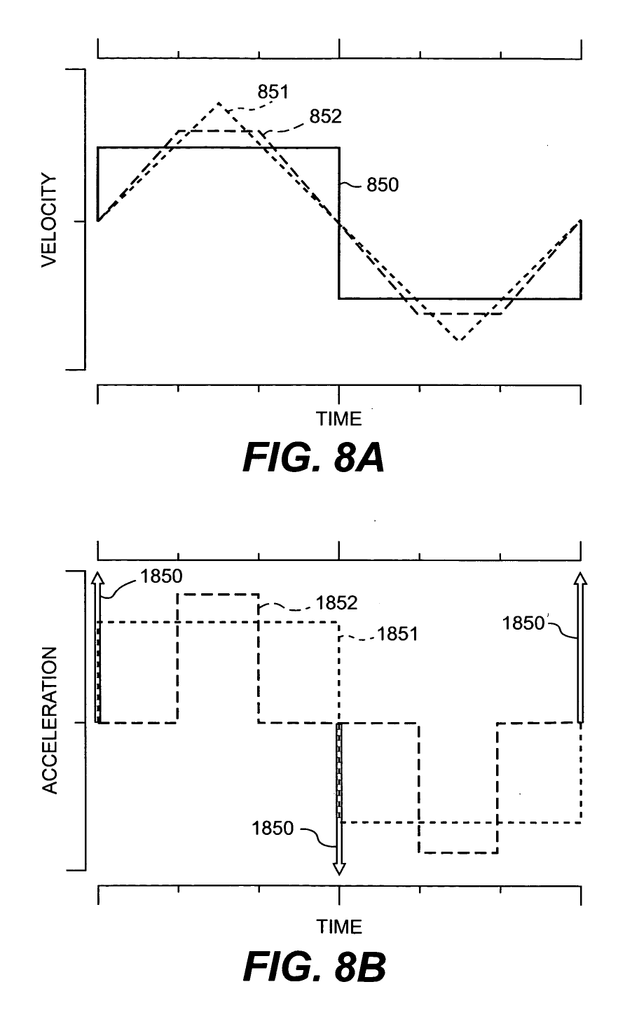

FIGS. 8A and 8B show three example profiles that can be used to control linear reciprocation of tray 110. FIGS. 8A and 8B show the velocity (FIG. 8A) and acceleration (FIG. 8B) for one period of a profile that contains a single fundamental frequency, where both boundary points of the period are shown for clarity (if a function has a period T, then time axis t for one period would be tₒ≦t<tₒ+T for any tₒ; for clarity the time axis has been extended to tₒ≦t≦tₒ+T). Profiles with multiple fundamental frequencies are also possible, where multiple fundamental frequencies can be separated in time (e.g., a first set of single or multiple fundamental frequencies followed by a second set of different single or multiple fundamental frequencies, etc., the number of sets being greater than 1) or superposed at the same time by adding the individual time waveforms together. Velocity profile 850 has a corresponding acceleration profile 1850. The large amplitude, short duration accelerations that accompany a step change in velocity are represented by impulses. Similarly, velocity profile 851 has a corresponding acceleration profile 1851 and velocity profile 852 has a corresponding acceleration profile 1852. The acceleration profiles are related to their respective velocity profiles by mathematical differentiation.

The three profiles shown in FIGS. 8A and 8B are all piecewise constant in either velocity or acceleration. Velocity profile 850 is piecewise constant with 2 piecewise constants having one positive and one negative value. While velocity profile 851 is not piecewise constant, the associated acceleration profile 1851 is piecewise constant with 2 piecewise constants having one positive value and one negative value. Acceleration profile 1852 is piecewise constant with 3 piecewise constants having one positive value, one negative value and one zero value. One skilled in the art can readily ascertain that many piecewise constant profiles can be generated, varying in the magnitude, number, and location of the piecewise constants as well as the time for one period. For example, the velocity profiles 850, 851, and 852 may be modified to have a constant zero velocity component at each point where the velocity crosses zero (i.e., when the reciprocation is changing directions). If drive mechanism 178 is a stepping motor, then small changes in the continuous-time velocity and acceleration profiles shown in FIG. 8A and FIG. 8B may occur due to the quantized step rate of the motor.

In one aspect, the controller 101 may be configured to control linear reciprocation of the tray to have either a piecewise constant velocity profile or a piecewise constant acceleration profile in which the number of piecewise constants does not exceed 24. According to another aspect, the number of piecewise constants does not exceed 12. In still another aspect the number of piecewise constants equals 3. While in yet another aspect, the number of piecewise constants equals 2. It should be appreciated that the computational complexity of generating the appropriate timing to drive a motor may be smaller when only the velocity and acceleration are controlled for a given displacement. This general-purpose motion control may need only minimal adaptation between moving the container from the extended position to inside the biological detection system and moving the container in an approximately sinusoidal manner. Furthermore, the amount of harmonic content in the agitation may be modified by selecting a velocity and/or acceleration that closely or more distantly approximates a sinusoid. During agitation, it may be desirable to minimize the accelerations that the rest of the detection system experiences during agitation and prevent the samples from splashing out of the container, while ensuring that the agitation achieves satisfactory mixing of the samples.

According to an aspect of the invention, the controller 101 may be configured to control linear reciprocation of the tray using a profile that is trapezoidal in shape, similar to velocity profile 852. According to one aspect, each wavelength of a trapezoidal profile includes increasing positive velocity component, a constant positive velocity component, a decreasing positive velocity component, a decreasing negative velocity component, a constant negative velocity component, and an increasing negative velocity component. According to one aspect, each of these 6 components is approximately equal in duration. According to one aspect, the linear reciprocation has a fundamental frequency of approximately 20 Hz, has an amplitude of approximately 3 mm, and has the 5^{th} harmonic being second only to the fundamental in amplitude.

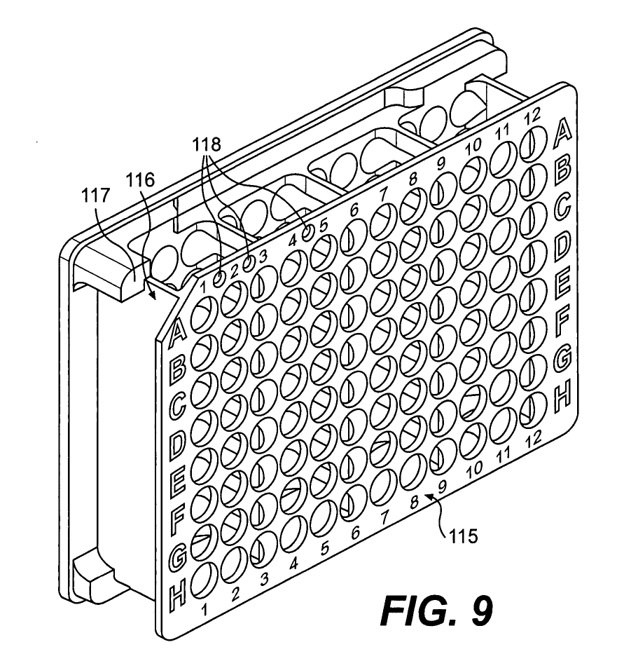

Turning now to FIG. 9, a container 115 may include a key used to ascertain correct orientation of the container 115 when placed in a detection system, for example, a biological detection system. For example, according to an exemplary aspect, a detection system may permit usage to custom containers comprising a subset of industry standard containers, for example, SBS-compliant microplates, while still permitting usage of non-custom, SBS-compliant microplates. As shown in FIG. 9, a non-symmetric key may be arranged on a standard container 115, for example, via a notch 116 or hole 118 feature, or via the depth 117 of such a feature. Then, using only pre-existing hardware in the biological detection system, for example, the probe 150, the motion control system 102, the controller 101, and/or a force sensor (not shown) on the probe 150, the may be interrogated for orientation, and additionally or alternatively, for the type of custom sample container. For example, in operation, the motion control system 102 may move the probe 150 to a preselected position associated with the key 116. Based on the position of the probe 150 and/or the presence or absence of force on the probe 150, the controller 101 may determine whether the custom container is correctly positioned and/or what type of custom container is present. If a non-custom container is used, this feature may be disabled or overridden to allow operation of the detection device.

A detection system may be used in a mobile environment where the system may be accidentally turned upside down or on its side. In these and other cases, having reagent and waste bottles that do not leak is advantageous. Because liquid is either being added or removed from these bottles, the quantity of air present in these bottles must change before a disadvantageous air pressure is created. Many systems use bottles that are vented with small holes to allow air to exchange, equalizing the pressure. Liquid can escape from these vent holes during a bottle inversion, particularly when the weight of the liquid over the vent hole is multiplied by an acceleration factor if the detection system is dropped. An apparatus to seal the bottles that is contained within the bottle or the lid of the bottle is advantageous because, for example, in the case of a biological detection system, the fraction of the system exposed to biologically hazardous materials is minimized.

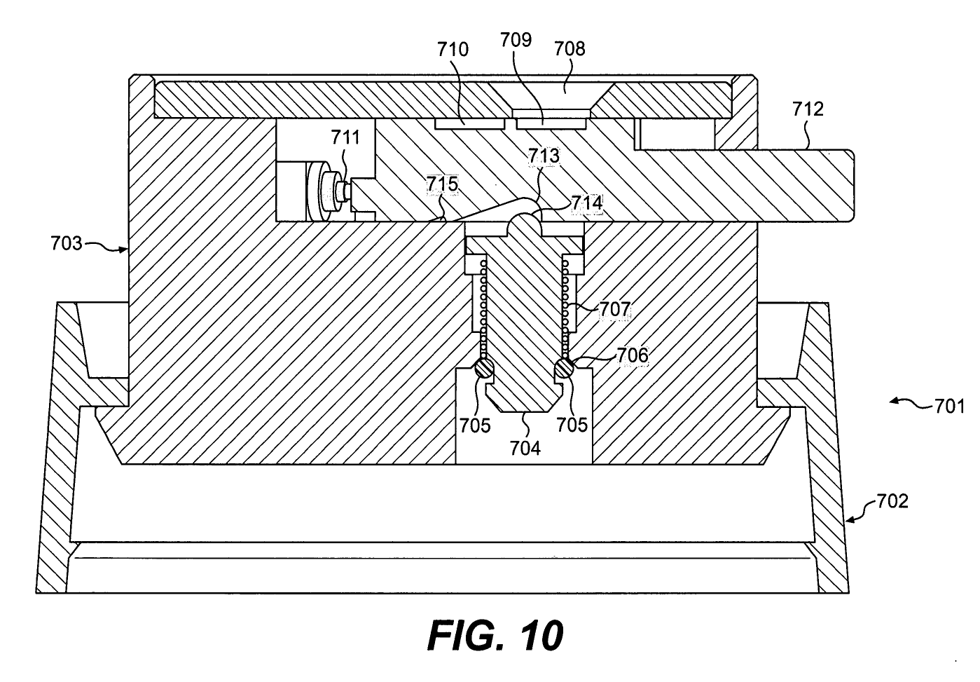

FIG. 10 shows a cap 701 to waste bottle 700 (FIG. 1). Venting mechanism 703 sits inside the threaded part of the cap 702 that screws onto a bottle. Sealing plunger 704 holds an o-ring 705 against sealing surface 706 using force generated by spring 707. In the shown configuration, the sealed symbol 709 is visible through indicator port 708, and switch 711 registers the sealed state electronically for the detection system. When an operator moves actuator 712 out, the open symbol 710 is visible through indicator port 708 and plunger tip 714 and plunger 704 are pushed down along actuator ramp 713, ending at actuator detent 715. In the open state, plunger 704 and o-ring 705 are pushed downwards, creating an air path between o-ring 705 and sealing surface 706. Further, in the open state, switch 711 registers the open state electronically for the detection system.

It will be apparent to those skilled in the art that various modifications and variations can be made in the disclosed detection device, components, and methods without departing from the scope of the invention. Other embodiments of the invention will be apparent to those skilled in the art from consideration of the specification and practice of the invention disclosed herein. It is intended that the specification and examples be considered as exemplary only, with a true scope of the invention being indicated by the following claims and their equivalents

1. An apparatus for retaining a container in a biological detection device, the container being configured to hold a plurality of at least one of a sample and a reagent, and the container having any one of a plurality of different predetermined flange heights, the apparatus comprising:
a first positioning block comprising a retractable first positioning arm and at least one retaining ledge on the first positioning arm; and
a second positioning block having at least one additional retaining ledge, the first and second positioning blocks being arranged to receive the container, and the first positioning arm being adapted to selectively apply a biasing force to the container to position the container under said at least one additional retaining ledge.

2. The apparatus of claim 1, wherein the second positioning block further comprises a retractable slide, the slide being configured to apply a second biasing force to the container in a direction substantially opposite to the biasing force.

3. The apparatus of claim 2, wherein the second biasing force is lesser in magnitude than the biasing force.

4. The apparatus of claim 3, wherein when the biasing force is removed, the second biasing force ejects the container from said at least one additional retaining ledge.

5. The apparatus of claim 4, wherein the second positioning block further comprises a retractable second positioning arm, at least one of the second plurality of retaining ledges being on the second positioning arm, and the second positioning arm being configured to apply a third biasing force to the container that is lesser in magnitude than the biasing force.

6. The apparatus of claim 1, further comprising:
a base member; and
a tray configured to translate in a first linear direction relative to the base member between a retracted position and an extended position, the tray including the first and second positioning blocks.

7. The apparatus of claim 6, wherein the first biasing force is removed when the tray is in the extended position.

8. The apparatus of claim 6, further comprising a driving mechanism configured to translate the tray in the first linear direction, the driving mechanism also being configured to reciprocate the tray in the first linear direction so as to agitate contents of the container.

9. The apparatus of claim 8, wherein the driving mechanism includes a motor.

10. The apparatus of claim 8, wherein the driving mechanism includes a stepping motor.

11. A biological detection system, comprising:
a base member;
a tray linearly movable with respect to the base member between an extended position and a retracted position;
a first sensor on the base member, the first sensor being configured to detect whether the tray is in the retracted position;
a second sensor on the base member, the second sensor being a sensor configured to detect whether the tray is holding a container; and
an apparatus configured to conduct electrochemiluminescence measurements.

12. The system of claim 11, wherein the tray includes a first indicator and a second indicator, the first sensor being configured to detect the first indicator when the tray is in the retracted position, and the second sensor being configured to detect the second indicator when the tray is holding a container.

13. The system of claim 12, wherein the first and second sensors include electro-optic sensors, and the first and second indicators include vanes extending from the tray.

14. The system of claim 11, wherein the tray includes a first indicator and a second indicator, the first sensor being configured to detect the first indicator when the tray is in the retracted position, and the second sensor being configured to detect the second indicator when the tray is not holding a container.

15. The system of claim 14, wherein the first and second sensors include electro-optic sensors, and the first and second indicators include vanes extending from the tray.

16. An apparatus for training a probe to locate at least one of a reagent and a sample, the apparatus comprising:
a first surface;
a probe having a probe axis, the probe being movable relative to the first surface;
a motion control system for controlling relative movement of the probe with respect to the first surface in at least a first direction along the probe axis, and at least a second direction not parallel to the probe axis;
a training object, at least part of which being electrically conductive, having a training surface and a member in contact with the first surface;
means for applying an electrical signal between the probe and the training object via the first surface; and
means for measuring a change in said electrical signal.

17. The apparatus of claim 16, wherein the measured change in said electrical signal results from a measurement of at least one of (i) a DC potential, (ii) an AC potential, (iii) a DC current, (iv) an AC current, (v) a DC charge, and (vi) an AC charge.

18. The apparatus of claim 16, further comprising at least one alignment feature on the training object sized in accordance with a fabrication tolerance of the apparatus,
wherein knowledge of at least one of a location and a size of the alignment feature in three or fewer dimensions is refined from an original fabrication tolerance by using (i) the motion control system to move at least one of the probe and training object, and (ii) the electrical signals generated when the probe and aspects of the alignment feature contact one another.

19. A biological detection system, comprising:
a base member;
a tray configured to translate along a linear dimension relative to the base member, the tray being configured to hold a container;
a driving mechanism configured to reciprocate the tray along the linear dimension so as to agitate contents of the container; and
an apparatus configured to conduct electrochemiluminescence measurements.

20. The system of claim 19, wherein the driving mechanism includes a motor having an output shaft, the system further comprising:
a belt associated with the output shaft and forming a linear drive path for the tray, the output shaft being arranged at a first end of the drive path;
a wheel associated with the belt at a second end of the drive path, the belt having two substantially parallel belt portions extending from the output shaft to the wheel, the tray being attached to one of said two belt portions; and
a counterweight mounted to the other of said two belt portions such that the counterweight is configured to linearly translate in a direction opposite to a translation direction of the tray.

21. The system of claim 20, wherein the counterweight's weight is greater than 70% of the weight of the tray and less than 120% of the weight of the sum of the tray and the maximum expected weight of the container with at least one of a reagent and sample.

22. The system of claim 20, wherein the counterweight is substantially the same weight as the tray.

23. The system of claim 19, wherein the driving mechanism reciprocates the tray in accordance with a trapezoidal motion profile, each wavelength of the profile having an increasing positive velocity component, a constant positive velocity component, a decreasing positive velocity component, a decreasing negative velocity component, a constant negative velocity component, and an increasing negative velocity component.

24. The system of claim 23, wherein said wavelength includes at least one constant zero velocity component.

25. The system of claim 23, wherein the six said components have approximately equal durations.

26. A biological detection system comprising:
a latching mechanism for a movable member, the movable member being configured to translate in a linear direction relative to a base member between a retracted position and an extended position, the latching mechanism comprising
a latching member configured to latch the movable member in the retracted position, the latching member being movable between a latching position and an unlatching position; and
a spring-biased member configured to urge the movable member in a direction away from the retracted position.

27. The biological detection system of claim 26, further comprising:
at least one additional movable member, said movable member and each of said at least one additional movable members being movable in a direction not parallel to one another; and
an additional latching mechanism associated with each additional movable member, each additional latching mechanism comprising
a latching member configured to latch the respective additional movable member in the retracted position, the latching member being movable between a latching position and an unlatching position; and
a spring-biased member configured to urge the respective additional movable member in a direction away from the retracted position.

28. The biological detection system of claim 26, further comprising:
at least one additional movable member, said movable member and each of said at least one additional movable members being movable in a direction not parallel to one another, said latching member being configured to latch one of said at least one additional movable member.

29. The biological detection system of claim 26, wherein the latching mechanism further comprises:
a solenoid configured to move the latching member between the latching position and the unlatching position.

30. A positive displacement pump comprising:
a reagent supply line;
a pump interface line from which the pump aspirates and dispenses fluid;
a storage line fluidly connectable with a pump chamber; and
means for selectively connecting the storage line to one of the reagent supply line and the pump interface line.

31. The pump of claim 30, wherein said means for selectively connecting the storage line is a 3-way valve.

32. The pump of claim 30, further comprising:
waste line; and
means for selectively connecting the pump chamber to one of the waste line and the storage line.

33. The pump of claim 32, wherein the means for selectively connecting the pump chamber is a 3-way valve.

34. A method of retaining a container in a biological detection device, the container having any one of a plurality of different predetermined flange heights, the method comprising:
retracting a first positioning arm;
placing the container in a tray;
translating the tray along a translation path;
engaging the container from a first direction with the first positioning arm;
engaging the container from a second direction with a second positioning block, the second direction being opposite to the first direction; and
applying a biasing force in the first direction to the container to position the container under at least one retaining ledge.

35. The method of claim 34, further comprising applying a second biasing force to the container in a direction opposite to the biasing force, the second biasing force being lesser in magnitude than the biasing force.

36. The method of claim 35, further comprising:
removing the biasing force, and
ejecting the container from said at least one retaining ledge via the second biasing force.

37. The method of claim 34 further comprising translating the tray between a retracted position and an extended position, the first positioning arm being retracted when the tray is in the extended position.

38. The method of claim 37, wherein the first biasing force is removed when the tray is in the extended position.

39. The method of claim 34, further comprising reciprocating the tray along a linear dimension so as to agitate contents of the container.

40. A method of determining a status of a movable tray, the method comprising:
detecting whether a tray is in a retracted position with a sensor on the base member; and
detecting whether the tray is holding a container with a sensor on the base member.

41. The method of claim 40, wherein said detecting whether a tray is in a retracted position includes detecting a first indicator extending from the tray when the tray is in the retracted position, and
wherein said detecting whether the tray is holding a container comprises detecting a second indicator extending from the tray when the tray is holding a container.

42. The method of claim 40, wherein said detecting whether a tray is in a retracted position includes detecting a first indicator extending from the tray when the tray is in the retracted position, and
wherein said detecting whether the tray is holding a container comprises detecting a second indicator extending from the tray when the tray is not holding a container.

43. A method of training a probe along a probe axis to locate at least one of a reagent and a sample within a biological detection device, the probe having a probe axis, the method comprising:
moving one of the probe and a training object along at least one additional axis, different from the probe axis, to within an initial estimate of an alignment feature; and
moving the probe along the probe axis into the alignment feature until the probe is sufficiently close to the training object that an electrical signal is generated.

44. The method of claim 43, wherein each of said at least one additional axis and said probe axis are not parallel to one another.

45. A method of training a probe along at least one axis not parallel to a probe axis to locate at least one of a reagent and a sample within a biological detection device, the method comprising:
moving one of the probe and a training object along at least one additional axis, different from the probe axis, so that the probe and the training object are within an initial estimate of an alignment feature along said at least one additional axis;
moving the probe along the probe axis into the alignment feature until the probe is below an uppermost surface of the alignment feature;
moving one of the probe and the training object along a training axis in a first direction and a second direction opposite to the first direction until the probe is sufficiently close to the training object in each of the first and second directions that electrical signals are generated; and
determining a center point of the alignment feature along the training axis.

46. A method of training a probe along at least two axes not parallel to a probe axis to locate at least one of a reagent and a sample within a biological detection device, the method comprising:
(i) moving one of the probe and a training object along all axes to be trained, different from the probe axis, so that the probe and the training object are object are within an initial estimate of an alignment feature along said axes to be trained;
(ii) moving the probe along the probe axis into the alignment feature until the probe is below an uppermost surface of the alignment feature;
(iii) moving one of the probe and training object along one of the axes to be trained alternately in both possible directions until the probe is sufficiently close to the training object in each of the directions that electrical signals are generated;
(iv) computing and then moving the probe to an estimate of the center point of the alignment feature
(v) repeating steps (iii) and (iv) for all axes to be trained; and
(vi) repeating step (v) until one of (a) the change in the estimate of the center point of the alignment feature is sufficiently small and (b) the desired number of iterations of (v) has occurred.

47. A biological detection method, comprising:
reciprocating a tray relative to a base member in a first linear direction so as to agitate contents of a container; and
conducting electrochemiluminescence measurements on at least one sample located in the container.

48. The method of claim 47, wherein said reciprocating comprises driving a belt with a drive mechanism, the method further comprising:
counterbalancing a weight of the tray with a counterweight coupled to a belt; and
reciprocating the counterweight in a second linear direction opposite to the first linear direction of the tray.

49. The method of claim 48, wherein the drive mechanism reciprocates the tray in accordance with a trapezoidal motion profile, each wavelength of the profile having an increasing positive velocity component, a constant positive velocity component, a decreasing positive velocity component, a decreasing negative velocity component, a constant negative velocity component, and an increasing negative velocity component.

50. The method of claim 49, wherein said wavelength includes at least one constant zero velocity component.

51. A method of latching a movable member in a biological detection system, the method comprising:
translating the movable member in a linear direction relative to a base member between a retracted position and an extended position;
latching the movable member in the retracted position; and
urging the latched movable member in a direction away from the retracted position.

52. A method of unlatching a movable member in a biological detection system, the method comprising:
moving the movable member in a direction away from the extended position;
moving the latching member from the latching position to the unlatching position; and
urging the movable member toward the extended position.

53. The method of claim 52, wherein said moving the movable member frees the latching mechanism to move from the latching position to the unlatching position.

54. A method of operating a positive displacement pump comprising:
selectively directing a flow of fluid from a reagent supply line to a storage line fluidly connectable to a pump chamber; and
selectively directing a flow of fluid from said storage line to a pump interface line from which the pump aspirates and dispenses fluid.

55. The method of claim 54, further comprising preventing said reagent directed to the storage line that is to be dispensed from the pump interface line from entering the pump chamber.

56. The method of claim 55, further comprising selectively directing fluid from the pump chamber to a waste line.

57. A biological detection system, comprising:
a base member;
a tray configured to translate in a first linear direction relative to the base member, the tray being configured to hold a container;
a driving mechanism configured to reciprocate the tray in the first linear direction so as to agitate contents of the container; and
a controller configured to control linear reciprocation of the tray to have one of a piecewise constant velocity profile and piecewise constant acceleration profile in which the number of piecewise constants does not exceed 24.

58. The system of claim 57, wherein the number of piecewise constants does not exceed 12.

59. The system of claim 57, wherein the number of piecewise constants equals 3.

60. The system of claim 57, wherein the number of piecewise constants equals 2.

61. A method of agitating samples in a biological detection system, comprising:
reciprocating a tray relative to a base member in a first linear direction so as to agitate contents of a container; and
controlling linear reciprocation of the tray to have one of a piecewise constant velocity profile and piecewise constant acceleration profile in which the number of piecewise constants does not exceed 24.

62. The method of claim 61, wherein the number of piecewise constants does not exceed 12.

63. The method of claim 61, wherein the number of piecewise constants equals 3.

64. The method of claim 61, wherein the number of piecewise constants equals 2.

65. A fluid handling station for a biological detection device, comprising:
a port configured to receive a probe;
a chamber extending from the port to a closed end, the chamber having a first portion connected to a second portion via a tapered region, the first portion having a cross-sectional area greater than that of the second portion; and
at least one fluid line configured to direct liquid reagent to the chamber, each of said at least one fluid line coupled to the chamber at substantially the same distance from the closed end and below the tapered region.

66. The fluid handling station of claim 65, further comprising:
an additional fluid line coupled to the chamber at a greater distance from the closed end than each of said at least one fluid line; and
a gas line coupled to the chamber at a greater distance from the closed end than each of said at least one fluid line and said additional fluid line.

67. A method of ascertaining correct orientation of a container in a biological detection device, the method comprising:
inserting a container into a biological detection device;
moving a probe used to aspirate and dispense fluids in the detection device to a predetermined location corresponding with a key associated with the container;
detecting whether the key is at the predetermined location; and
determining, based on said detecting, whether the container is correctly oriented in the detection system.

68. The method of claim 67, further comprising:
determining, based on said detecting, a type of container inserted in the detection system.

69. An apparatus for venting one of a reagent bottle and a waste bottle in a biological detection device, the apparatus comprising:
a two-state sealing mechanism built into one of the bottle and the bottle cap, the two states being (a) to connect the interior space in the bottle to exterior and (b) to close said connection; and
an indicating mechanism to unambiguously indicate the state of the sealing mechanism.

70. The apparatus of claim 69 wherein the indicating mechanism is visual.

71. The apparatus of claim 69, where the indicating mechanism is an electrical signal that is fed back to another aspect of the biological detection system.

72. The apparatus of claim 16, further comprising at least one alignment feature on the training object sized in accordance with a fabrication tolerance of the apparatus,
wherein knowledge of at least one of a location and a size of the alignment feature in three or fewer dimensions is refined from an original fabrication tolerance by using (i) the motion control system to move at least one of the probe and training object, and (ii) the electrical signals generated when the probe and aspects of the alignment feature are in close proximity to one another.

73. The system of claim 19, wherein the driving mechanism includes:
a motor having an output shaft;
a bearing mounted on the output shaft; and
a power transfer mechanism mounted on the output shaft, the bearing position being closer to a body of the motor than the power transfer mechanism.

74. The system of claim 73, wherein the bearing resists greater than 50% of the linear force applied to the motor shaft via the power-transfer mechanism.

75. A method of loosening and re-tightening a motor on a mounting so as to transfer a majority of a load on a shaft of the motor to an external bearing, the external bearing being located between the load and the motor.

76. A method of training a probe along a probe axis to locate at least one of a reagent and a sample within a biological detection device, the probe having a probe axis, the method comprising:
moving one of the probe and a training object along at least one additional axis, different from the probe axis, to the center point as determined by the method of claim 45; and
moving the probe along the probe axis into the alignment feature until the probe is sufficiently close to a training object that an electrical signal is generated.

77. A method of training a probe along a probe axis to locate at least one of a reagent and a sample within a biological detection device, the probe having a probe axis, the method comprising:
moving one of the probe and a training object along at least one additional axis, different from the probe axis, to the center point as determined by the method of claim 46; and
moving the probe along the probe axis into the alignment feature until the probe is sufficiently close to a training object that an electrical signal is generated,

78. The method of claim 49, wherein the six said components have approximately equal durations.

79. The method of claim 51, wherein said method occurs with one of (i) electrical power using the biological detection system's controller, and (ii) without electrical power using the biological detection system's operator, wherein the operator is not required to use tools.

80. The system of any of claims 57-60, further comprising:
an apparatus configured to conduct electrochemiluminescence measurements.

81. The method of any of claims 61-64, further comprising:
conducting electrochemiluminescence measurements on at least one sample located in a container.
